(19) United States
(12) Patent Application Publication
(10) Pub. No.: US 2015/0111864 A1
(43) Pub. Date: Apr. 23, 2015

(54) CRYSTALLINE FORM OF A BETA-LACTAMASE INHIBITOR

(71) Applicant: Cubist Pharmaceuticals, Inc., Lexington, MA (US)

(72) Inventors: Sudhakar Garad, Malden, MA (US); Akash Jain, Burlington, MA (US); You Seok Hwang, Windham, NH (US)

(21) Appl. No.: 14/504,786

(22) Filed: Oct. 2, 2014

Related U.S. Application Data
(60) Provisional application No. 61/885,730, filed on Oct. 2, 2013.

(51) Int. Cl. C07D 471/08; A61K 31/545; A61K 31/439

(52) U.S. Cl. CPC C07D 471/08; A61K 31/439; A61K 31/545
USPC 514/202; 546/121; 514/300

(57) ABSTRACT

This disclosure provides compositions containing solid forms of (2S,5R)-2-(5-(3-aminopropyl)-1,3,4-oxadiazol-2-yl)-7-oxo-1,6-diazabicyclo[3.2.1]octan-6-yl hydrogen sulfate, and methods of manufacturing and using these compositions.

### CROSS-REFERENCE TO RELATED APPLICATIONS

This application claims priority to U.S. Provisional Application No. 61/885,730, filed Oct. 2, 2013. The contents of this application are incorporated hereby by reference in their entirety.

### TECHNICAL FIELD

This disclosure relates to crystalline solid forms of (2S,5R)-2-(5-(3-aminopropyl)-1,3,4-oxadiazol-2-yl)-7-oxo-1,6-diazabicyclo[3.2.1]octan-6-yl hydrogen sulfate, and related compositions and methods.

### BACKGROUND

The solid state of a compound can be important when the compound is used for pharmaceutical purposes. Crystalline solid forms of a compound may exhibit properties that are different from one another, or different from amorphous forms of the compound. The solid physical properties of a compound can change from one form to another, which can affect the suitability of the form for pharmaceutical use. For example, a particular crystalline solid compound can overcome the disadvantage of other solid forms of the compound that are unstable (e.g., that convert from one solid form to another).

Provided herein is a solid crystalline form of (2S,5R)-2-(5-(3-aminopropyl)-1,3,4-oxadiazol-2-yl)-7-oxo-1,6-diazabicyclo[3.2.1]octan-6-yl hydrogen sulfate, which exhibits improved stability properties that are advantageous in drug substance and drug product development.

### SUMMARY

Provided herein are crystalline forms of the compound of Formula (I):

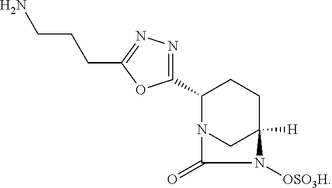

In one embodiment, the crystalline form is characterized by an X-ray powder diffraction pattern having four or more peaks expressed in degrees 2-theta at angles of 9.0°±0.2°, 10.0°±0.2°, 10.8°±0.2°, 13.3°±0.2°, 16.0°±0.2°, 19.7°±0.2°, 22.5°±0.2°, 28.3°±0.2°, and 29.3°±0.2°. In another embodiment, the form is characterized by an X-ray powder diffraction pattern having peaks expressed in degrees 2-theta at angles of 9.0°±0.2°, 10.0°±0.2°, 22.5°±0.2° and 29.3°±0.2°.

In yet another embodiment, the crystalline form is characterized by an X-ray powder diffraction pattern having four or more peaks expressed in degrees 2-theta at angles of 9.0±0.2°, 10.0±0.2°, 14.0±0.2°, 17.9±0.2°, 20.1±0.2°, 21.0±0.2°, 22.5±0.2°, 24.3±0.2°, and 29.3±0.2°. In still another embodiment, the form is characterized by an X-ray powder diffraction pattern having peaks expressed in degrees 2-theta at angles of 9.0±0.2°, 10.0±0.2°, 20.1±0.2°, 21.0±0.2°, and 22.5±0.2°.

In another aspect, provided herein is a pharmaceutical composition comprising a crystalline form of the compound of Formula (I) and optionally a pharmaceutically acceptable carrier or diluent. In one embodiment, the pharmaceutical composition further comprises at least one β-lactam antibiotic. In a particular embodiment, the β-lactam antibiotic is a cephalosporin. In a more particular embodiment, the cephalosporin is Ceftolozane. In another particular embodiment, the β-lactam antibiotic is a carbapenem. In yet another particular embodiment, the β-lactam antibiotic is a monobactam.

In another aspect, provided herein is a method of treating or preventing a bacterial infection comprising administering to a subject in need thereof a therapeutically-effective amount of a crystalline form of the compound of Formula (I) or a pharmaceutical composition thereof.

In another aspect, provided herein is a method of treating or preventing a bacterial infection comprising administering to a subject in need thereof a therapeutically-effective amount of crystalline forms, amorphous forms, or mixture of solid forms of the compound of Formula (I) or a pharmaceutical composition thereof. Other embodiments provide for obtaining a pharmaceutical composition by dissolving a crystalline form of the compound of Formula (I) in solution, and obtaining a solid pharmaceutical composition from the solution (e.g., by lyophilizing the solution). The solid pharmaceutical composition can be subsequently reconstituted in a pharmaceutically acceptable diluent prior to intravenous administration.

In another aspect, provided herein is a method of treating or preventing a bacterial infection comprising administering to a subject in need thereof a therapeutically-effective amount of a β-lactam antibiotic in conjunction with a crystalline form of the compound of Formula (I).

In another aspect, provided herein is a method of treating a bacterial infection in a subject in need thereof, comprising the steps of:

(I) administering to the subject a therapeutically-effective amount of a β-lactam antibiotic; then

(II) administering to the subject a crystalline form of Formula (I).

In another aspect, provided herein is a method of treating a bacterial infection in a subject in need thereof, comprising the steps of:

(a) administering to the subject a crystalline form of Formula (I); then

(b) administering to the subject a therapeutically-effective amount of a β-lactam antibiotic.

In some embodiments of these methods, the bacterial infection is caused by bacteria that produce a class A, class C or class Dβ-lactamase. In some embodiments, the bacterial infection is caused by bacteria that produce a class A or class Cβ-lactamase. In other embodiments, the bacterial infection is caused by bacteria selected from Acinetobacter spp., Acinetobacter baumannii, Citrobacter spp., Escherichia spp., Escherichia coli, Haemophilus influenzae, Morganella morganii, Pseudomonas aeruginosa, Klebsiella spp., Klebsiella pneumoniae, Enterobacter spp., Enterobacter cloacae, Enterobacter aerogenes Pasteurella spp., Proteus spp., Proteus mirabilis, Serratia spp., Serratia marcescens, and Providencia spp. In a particular embodiment, the bacterial infection is caused by bacteria that produce a KPC-2 or KPC-3 β-lactamase. In another particular embodiment, the bacterial infection is caused by bacteria that produce an OXA-15 β-lactamase. In yet another particular embodiment, the bacterial infection is caused by β-lactam resistant bacteria.

In another aspect, provided herein is a method of inhibiting β-lactamase comprising administering to a subject a crystalline form of Formula (I).

In another aspect, provided herein is a method of making a crystalline form of the compound of Formula (I), comprising the steps of: (1) combining (2S,5R)-2-(5-(3-aminopropyl)-1,3,4-oxadiazol-2-yl)-7-oxo-1,6-diazabicyclo[3.2.1]octan-6-yl hydrogen sulfate and a solvent, such that a solution of (2S,5R)-2-(5-(3-aminopropyl)-1,3,4-oxadiazol-2-yl)-7-oxo-1,6-diazabicyclo[3.2.1]octan-6-yl hydrogen sulfate is formed; and (2) evaporating the solvent, such that a crystalline solid form of the compound of Formula (I) is formed.

In another aspect, provided herein is a method of making a crystalline form of the compound of Formula (I), comprising the steps of: (1) combining (2S,5R)-2-(5-(3-aminopropyl)-1,3,4-oxadiazol-2-yl)-7-oxo-1,6-diazabicyclo[3.2.1]octan-6-yl hydrogen sulfate and a solvent, such that a first solution of (2S,5R)-2-(5-(3-aminopropyl)-1,3,4-oxadiazol-2-yl)-7-oxo-1,6-diazabicyclo[3.2.1]octan-6-yl hydrogen sulfate is formed; (2) combining the first solution with a second solvent; and (3) crystallizing the compound of Formula (I).

In some embodiments of the above methods, the crystalline form of the compound of Formula (I) is characterized by an X-ray powder diffraction pattern having four or more peaks expressed in degrees 2-theta at angles of 9.0°±0.2°, 10.0°±0.2°, 10.8°±0.2°, 13.3°±0.2°, 16.0°±0.2°, 19.7°±0.2°, 22.5°±0.2°, 28.3°±0.2°, and 29.3°±0.2°.

In other embodiments of the above methods, the crystalline form of the compound of Formula (I) is characterized by an X-ray powder diffraction pattern having four or more peaks expressed in degrees 2-theta at angles of 9.0±0.2°, 10.0±0.2°, 14.0±0.2°, 17.9±0.2°, 20.1±0.2°, 21.0±0.2°, 22.5±0.2°, 24.3±0.2°, and 29.3±0.2°.

In another aspect, provided herein is the use of a crystalline form of the compound of Formula (I), or a pharmaceutical composition thereof, in the manufacture of a medicament for treating bacterial infections.

### BRIEF DESCRIPTION OF THE DRAWINGS

FIG. 1 depicts the X-ray powder diffraction pattern of Form 5 of the compound of Formula (I).

FIG. 2 depicts XRPD patterns of Form 5 of the compound of Formula (I) t=0 and t=2 days.

FIG. 3 depicts the thermogravimetry (TGA) curve (upper) and the differential scanning calorimetry (DSC) curve (lower) of Form 5.

FIG. 4 depicts the DVS graph of Form 5.

FIG. 5 depicts the X-ray powder diffraction patterns of a number of crystalline forms of the compound of Formula (I) identified in the polymorph studies described herein.

FIG. 6 shows a diagram that summarizes the relative thermodynamic relationships between the different solid forms of the compound of Formula (I).

FIG. 7 shows the single crystal X-ray structure of Form 5.

FIG. 8 shows a representative temperature/turbidity plot for Form 5.

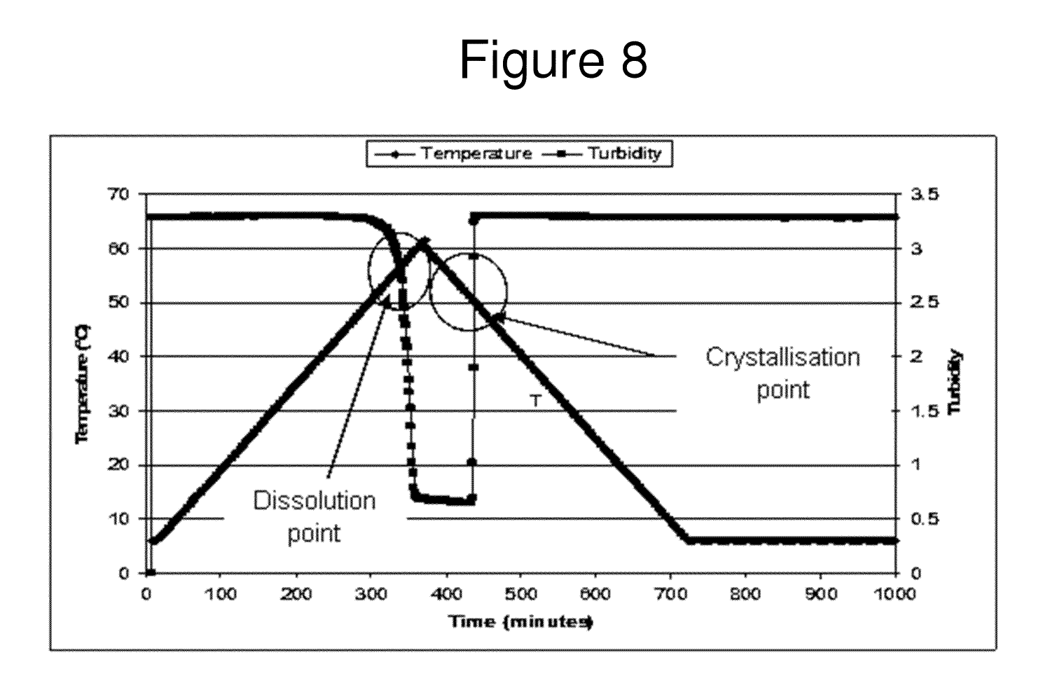

### DETAILED DESCRIPTION

Bacterial resistance to β-lactam antibiotics, especially in Gram-negative bacteria, is most commonly mediated by β-lactamases. β-lactamases are enzymes that catalyze the hydrolysis of the β-lactam ring, which inactivates the antibacterial activity of the β-lactam antibiotic and allows the bacteria to become resistant. Inhibition of the β-lactamase with a β-lactamase inhibitor (BLI) slows or prevents degradation of the β-lactam antibiotic and restores β-lactam antibiotic susceptibility to β-lactamase producing bacteria. The compound of Formula (I) (i.e., (2S,5R)-2-(5-(3-aminopropyl)-1,3,4-oxadiazol-2-yl)-7-oxo-1,6-diazabicyclo[3.2.1]octan-6-yl hydrogen sulfate) is an effective BLI.

The compound of Formula (I) can occur in one or more amorphous solid forms or in crystalline solid forms. Crystalline solid forms of the compound of Formula (I) can exist in one or more unique polymorph forms. Accordingly, provided herein are crystalline solid forms of the compound of Formula (I) (i.e., (2S,5R)-2-(5-(3-aminopropyl)-1,3,4-oxadiazol-2-yl)-7-oxo-1,6-diazabicyclo[3.2.1]octan-6-yl hydrogen sulfate), and compositions comprising these crystalline solid forms, in addition to various methods of preparing these compositions.

A particular crystalline solid form of the compound of Formula (I) provided herein has advantageous characteristics that are beneficial to the preparation of various drug formulations. For example, a particular crystalline form of the compound of Formula (I) (i.e., Form 5) is more stable than other crystalline forms. This property is associated with good stability in the process of preparation, packing, transportation, and storage.

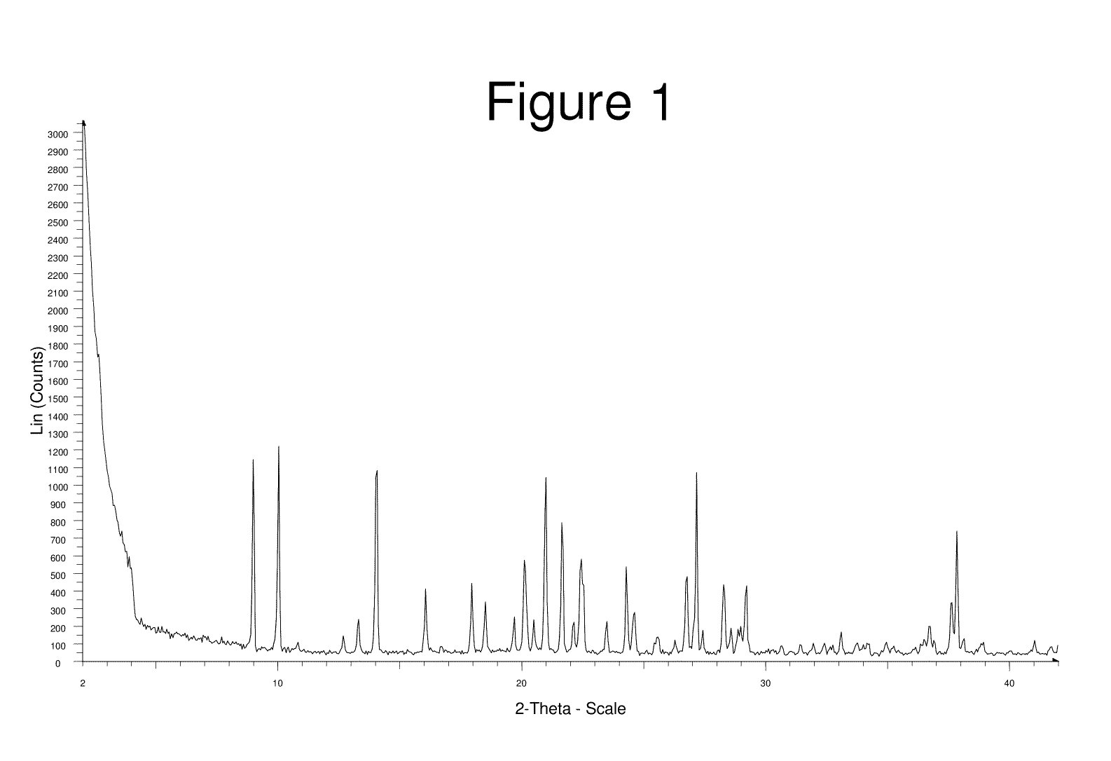

The compound of Formula (I) can be obtained in various solid crystalline forms. A preferred solid crystalline form of the compound of Formula (I) is provided, and is referred to herein as “the compound of Formula (I) Form 5”, “Form 5 of the compound of Formula (I)” or “Form 5”. In one embodiment, Form 5 is identified by a characteristic X-ray powder diffraction (XRPD) pattern (FIG. 1). In another embodiment, Form 5 is identified by an X-ray powder diffraction pattern having four or more peaks expressed in degrees 2-theta at angles of 9.0°±0.2°, 10.0°±0.2°, 10.8°±0.2°, 13.3°±0.2°, 16.0°±0.2°, 19.7°±0.2°, 22.5°±0.2°, 28.3°±0.2°, and 29.3°±0.2°. In another embodiment, Form 5 is identified by an X-ray powder diffraction pattern having four or more peaks expressed in degrees 2-theta at angles of 9.0±0.2°, 10.0±0.2°, 14.0±0.2°, 17.9±0.2°, 20.1±0.2°, 21.0±0.2°, 22.5±0.2°, 24.3±0.2°, and 29.3±0.2°.

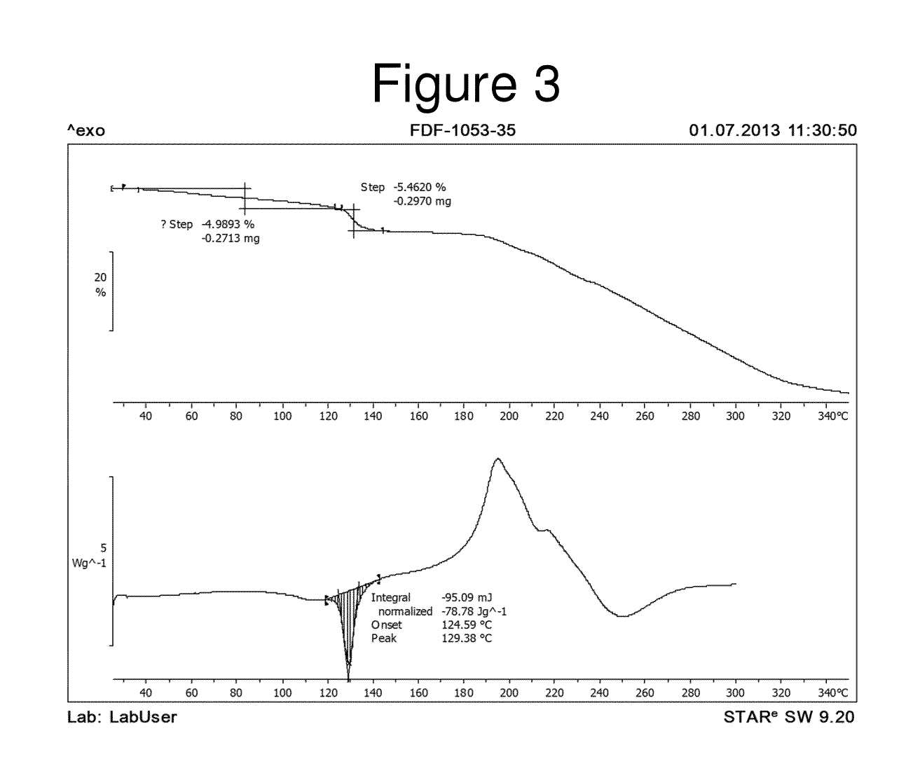

Form 5 can be further characterized by a differential scanning calorimetry (DSC) and by by thermogravimetric analysis (TGA). Both the DSC thermogram and the TGA curve are shown in FIG. 3.

### Polymorphism

The ability of a substance to exist in one or more than one solid form is defined as polymorphism; the different crystal forms of a particular substance are referred to as “polymorphs.” In general, polymorphism is affected by the ability of a molecule of a substance to change its conformation or to form different intermolecular or intramolecular interactions, particularly hydrogen bonds, which is reflected in different atom arrangements in the crystal lattices of different polymorphs. In contrast, the overall external form of a substance is known as “morphology,” which refers to the external shape of the crystal and the planes present, without reference to the internal structure. Crystals can display different morphology based on different conditions, such as, for example, growth rate, stirring, and the presence of impurities.

The different polymorphs of a substance can possess different energies of the crystal lattice and, thus, in solid state they can show different physical properties such as form, density, melting point, color, stability, solubility, dissolution rate, etc., which can, in turn, affect the stability, dissolution rate, and/or bioavailability of a given polymorph and its suitability for use as a pharmaceutical and in pharmaceutical compositions.

Access to different polymorphs of the compound of Formula (I) is desirable for other reasons as well. One such reason is that different polymorphs of a compound (e.g., the compound of Formula (I)) can incorporate different impurities, or chemical residues, upon crystallization. Certain polymorphs incorporate very little, or no, chemical residues. Accordingly, the formation of certain polymorph forms of a compound may result in purification of the compound.

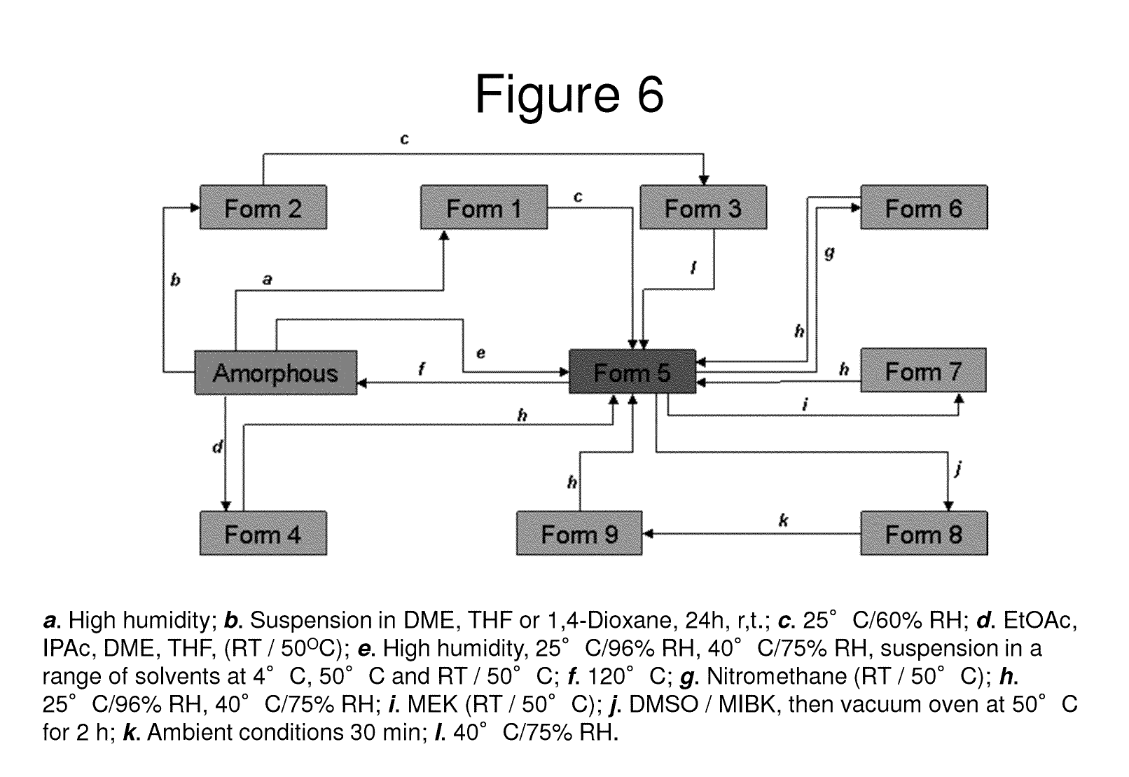

Form 5 of the compound of Formula (I) exhibits improved stability relative to the amorphous solid form, as well as other solid crystalline forms of the compound of Formula (I). As shown in FIG. 6, the amorphous solid form and other solid crystalline forms all convert to Form 5.

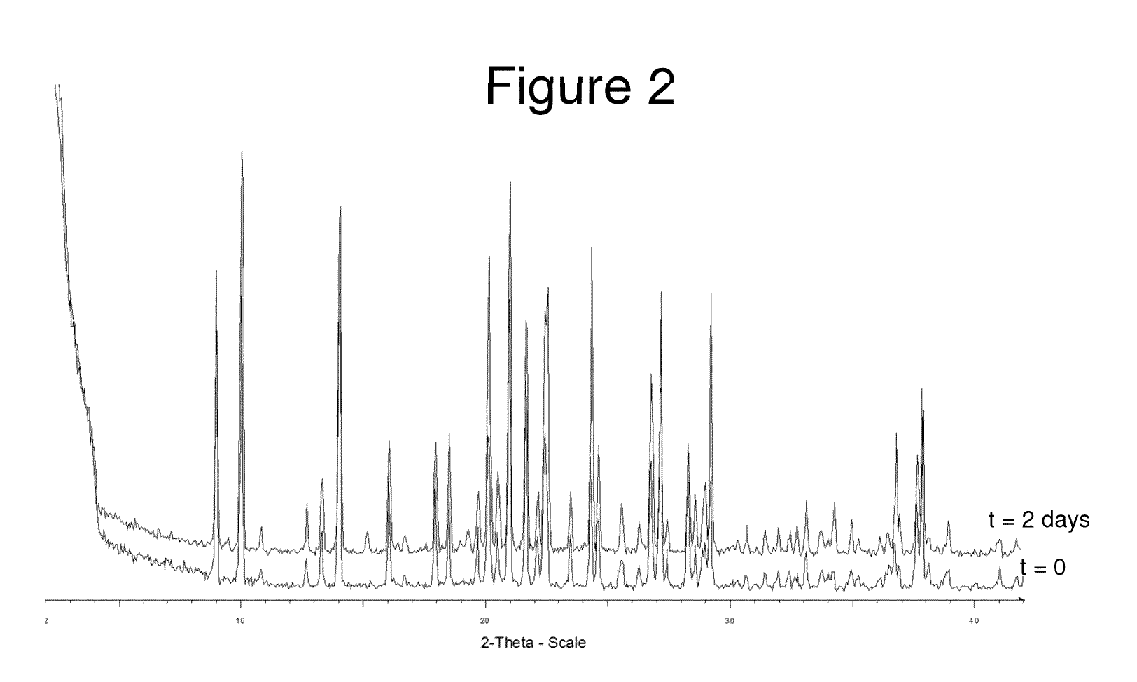

Moreover, Form 5 also demonstrated favorable stability over the course of time. For example, FIG. 2 depicts XRPD patterns of Form 5 (25° C. and 40% relative humidity) taken at t=0 and t=2 days. As shown in this figure, there is little to no change to the Form 5 crystal form over this time period.

### Characterization of Polymorphs

In certain embodiments, the compounds of the invention are identifiable on the basis of characteristic peaks in an X-ray powder diffraction analysis. X-ray powder diffraction (XRPD) is a scientific technique using X-ray, neutron, or electron diffraction on powder, microcrystalline, or other solid materials for structural characterization of solid materials.

Provided herein is a solid crystalline form of the compound of Formula (I), referred to as Form 5. In one embodiment, Form 5 is characterized by an X-ray powder diffraction pattern having four or more peaks expressed in degrees 2-Theta at angles selected from 9.0±0.2°, 10.0±±0.2°, 10.8±0.2°, 13.3±0.2°, 16.0±0.2°, 19.7±0.2°, 22.5±0.2°, 28.3±0.2°, and 29.3±0.2° (also referred to herein as “the group A peaks”). In another embodiment, Form 5 is characterized by an X-ray powder diffraction pattern having five or more peaks expressed in degrees 2-Theta at angles selected from the group A peaks. In another embodiment, Form 5 is characterized by an X-ray powder diffraction pattern having six or more peaks expressed in degrees 2-Theta at angles selected from the group A peaks. In another embodiment, Form 5 is characterized by an X-ray powder diffraction pattern having seven or more peaks expressed in degrees 2-Theta at angles selected from the group A peaks. In another embodiment, Form 5 is characterized by an X-ray powder diffraction pattern having eight or more peaks expressed in degrees 2-Theta at angles selected from the group A peaks. In another embodiment, Form 5 is characterized by an X-ray powder diffraction pattern having nine peaks expressed in degrees 2-Theta at angles selected from the group A peaks.

In another embodiment, Form 5 is characterized by an X-ray powder diffraction pattern having peaks expressed in degrees 2-Theta at angles of 9.0±0.2°, 10.0±0.2°, 22.5±0.2°, and 29.3±0.2°. In another embodiment, Form 5 is characterized by an X-ray powder diffraction pattern having peaks expressed in degrees 2-Theta at angles of 9.0±0.2°, 10.0±0.2°, 16.0±0.2°, 22.5±0.2°, and 29.3±0.2°. In another embodiment, Form 5 is characterized by an X-ray powder diffraction pattern having peaks expressed in degrees 2-Theta at angles of 9.0±0.2°, 10.0±0.2°, 16.0±0.2°, 22.5±0.2°, 28.3±0.2°, and 29.3±0.2°.

In another embodiment, Form 5 is characterized by an X-ray powder diffraction pattern having four or more peaks expressed in degrees 2-Theta at angles selected from 9.0±0.1°, 10.0±0.1°, 10.8±0.1°, 13.3±0.1°, 16.0±0.1°, 19.7±0.1°, 22.5±0.1°, 28.3±0.1°, and 29.3±0.1° (also referred to herein as the group B peaks). In another embodiment, Form 5 is characterized by an X-ray powder diffraction pattern having five or more peaks expressed in degrees 2-Theta at angles selected from the group B peaks. In another embodiment, Form 5 is characterized by an X-ray powder diffraction pattern having six or more peaks expressed in degrees 2-Theta at angles selected from the group B peaks. In another embodiment, Form 5 is characterized by an X-ray powder diffraction pattern having seven or more peaks expressed in degrees 2-Theta at angles selected from the group B peaks. In another embodiment, Form 5 is characterized by an X-ray powder diffraction pattern having eight or more peaks expressed in degrees 2-Theta at angles selected from the group B peaks. In another embodiment, Form 5 is characterized by an X-ray powder diffraction pattern having nine peaks expressed in degrees 2-Theta at angles selected from the group B peaks.

In yet another embodiment, Form 5 is characterized by an X-ray powder diffraction pattern having peaks expressed in degrees 2-Theta at angles of 9.0±0.1°, 10.0±0.1°, 22.5±0.1°, and 29.3±0.1°. In another embodiment, Form 5 is characterized by an X-ray powder diffraction pattern having peaks expressed in degrees 2-Theta at angles of 9.0±0.1°, 10.0±0.1°, 16.0±0.1°, 22.5±0.1°, and 29.3±0.1°. In another embodiment, Form 5 is characterized by an X-ray powder diffraction pattern having peaks expressed in degrees 2-Theta at angles of 9.0±0.1°, 10.0±0.1°, 16.0±0.1°, 22.5±0.1°, 28.3±0.1°, and 29.3±0.1°.

In still another embodiment, Form 5 is characterized by an X-ray powder diffraction pattern having peaks expressed in degrees 2-Theta at angles of 9.0°, 10.0°, 22.5°, and 29.3°. In another embodiment, Form 5 is characterized by an X-ray powder diffraction pattern having peaks expressed in degrees 2-Theta at angles of 9.0°, 10.0°, 16.0°, 22.5°, and 29.3°. In another embodiment, Form 5 is characterized by an X-ray powder diffraction pattern having peaks expressed in degrees 2-Theta at angles of 9.0°, 10.0°, 16.0°, 22.5°, 28.3°, and 29.3°. In another embodiment, Form 5 is characterized by an X-ray powder diffraction pattern having peaks expressed in degrees 2-Theta at angles of 9.0°, 10.0°, 10.8°, 13.3°, 16.0°, 19.7°, 22.5°, 28.3°, and 29.3°.

In yet another embodiment, Form 5 is characterized by an X-ray powder diffraction pattern having peaks expressed in degrees 2-Theta at angles of 10.0±0.2°, 20.1±0.2°, 21.0±0.2°, and 22.5±0.2°. In another embodiment, Form 5 is characterized by an X-ray powder diffraction pattern having peaks expressed in degrees 2-Theta at angles of 9.0±0.2°, 10.0±0.2°, 20.1±0.2°, 21.0±0.2°, and 22.5±0.2°. In still another embodiment, Form 5 is characterized by an X-ray powder diffraction pattern having peaks express in degrees 2-Theta at angles of 9.0±0.2°, 10.0±0.2°, 14.0±0.2°, 17.9±0.2°, 20.1±0.2°, 21.0±0.2°, 22.5±0.2°, 24.3±0.2°, and 29.3±0.2°.

In another embodiment, Form 5 is characterized by an X-ray powder diffraction pattern having four or more peaks expressed in degrees 2-Theta at angles selected from 9.0±0.2°, 10.0±0.2°, 14.0±0.2°, 17.9±0.2°, 20.1±0.2°, 21.0±0.2°, 22.5±0.2°, 24.3±0.2°, and 29.3±0.2° (also referred to herein as “the group C peaks”). In another embodiment, Form 5 is characterized by an X-ray powder diffraction pattern having five or more peaks expressed in degrees 2-Theta at angles selected from the group C peaks. In another embodiment, Form 5 is characterized by an X-ray powder diffraction pattern having six or more peaks expressed in degrees 2-Theta at angles selected from the group C peaks. In another embodiment, Form 5 is characterized by an X-ray powder diffraction pattern having seven or more peaks expressed in degrees 2-Theta at angles selected from the group C peaks. In another embodiment, Form 5 is characterized by an X-ray powder diffraction pattern having eight or more peaks expressed in degrees 2-Theta at angles selected from the group C peaks. In another embodiment, Form 5 is characterized by an X-ray powder diffraction pattern having nine peaks expressed in degrees 2-Theta at angles selected from the group C peaks.

In one embodiment, Form 5 is characterized by an X-ray powder diffraction pattern having peaks substantially in accordance with FIG. 1. In another embodiment, Form 5 is characterized by an X-ray powder diffraction pattern having peaks substantially in accordance with Table 1.

The crystalline Form 5 can be further characterized by a differential scanning calorimetry (DSC) thermogram having an endotherm with an onset temperature of 125° C. (see FIG. 3). In another embodiment, Form 5 is further characterized by a characteristic thermogravimetry (TGA) curve. As shown in FIG. 3, the first endothermic event occurs between 25° C.-125° C., with a corresponding weight loss of 4.99%. The second endothermic event occurs between 125° C.-145° C., with a corresponding weight loss of 5.46%.

In certain embodiments, Form 5 of the compound of Formula (I) can contain impurities. Non-limiting examples of impurities include undesired polymorph forms, or residual organic and inorganic molecules such as solvents, water or salts.

In one embodiment, Form 5 of the compound of Formula (I) is substantially free from impurities. In another embodiment, Form 5 contains less than 10% by weight total impurities. In another embodiment, Form 5 contains less than 5% by weight total impurities. In another embodiment, Form 5 contains less than 1% by weight total impurities. In yet another embodiment, Form 5 contains less than 0.1% by weight total impurities.

In certain embodiments, Form 5 is a crystalline solid substantially free of amorphous (2S,5R)-2-(5-(3-aminopropyl)-1,3,4-oxadiazol-2-yl)-7-oxo-1,6-diazabicyclo[3.2.1]octan-6-yl hydrogen sulfate. As used herein, the term “substantially free of amorphous (2S,5R)-2-(5-(3-aminopropyl)-1,3,4-oxadiazol-2-yl)-7-oxo-1,6-diazabicyclo[3.2.1]octan-6-yl hydrogen sulfate” means that the compound contains no significant amount of amorphous (2S,5R)-2-(5-(3-aminopropyl)-1,3,4-oxadiazol-2-yl)-7-oxo-1,6-diazabicyclo[3.2.1]octan-6-yl hydrogen sulfate. In certain embodiments, at least about 95% by weight of crystalline solid Form 5 of the compound of Formula (I) is present. In still other embodiments of the invention, at least about 99% by weight of crystalline solid Form 5 of the compound of Formula (I) is present.

In another embodiment, Form 5 is substantially free of polymorph Forms 1, 2, 3, 4, 6, 7, 8, or 9 of the compound of Formula (I). As used herein, the term “polymorph Forms 1, 2, 3, 4, 6, 7, 8, or 9” means that Form 5 contains no significant amount of polymorph Forms 1, 2, 3, 4, 6, 7, 8, or 9. In certain embodiments, at least about 95% by weight of crystalline solid Form 5 is present. In still other embodiments of the invention, at least about 99% by weight of crystalline solid Form 5 is present.

### Processes and Methods

Provided herein is a method of making crystalline (2S,5R)-2-(5-(3-aminopropyl)-1,3,4-oxadiazol-2-yl)-7-oxo-1,6-diazabicyclo[3.2.1]octan-6-yl hydrogen sulfate, comprising:

(1) combining (2S,5R)-2-(5-(3-aminopropyl)-1,3,4-oxadiazol-2-yl)-7-oxo-1,6-diazabicyclo[3.2.1]octan-6-yl hydrogen sulfate and a solvent, such that a solution of (2S,5R)-2-(5-(3-aminopropyl)-1,3,4-oxadiazol-2-yl)-7-oxo-1,6-diazabicyclo[3.2.1]octan-6-yl hydrogen sulfate is formed; and

(2) evaporating the solvent, such that crystalline (2S,5R)-2-(5-(3-aminopropyl)-1,3,4-oxadiazol-2-yl)-7-oxo-1,6-diazabicyclo[3.2.1]octan-6-yl hydrogen sulfate is formed.

In another embodiment, provided herein is a method of making Form 5, comprising:

(1) combining (2S,5R)-2-(5-(3-aminopropyl)-1,3,4-oxadiazol-2-yl)-7-oxo-1,6-diazabicyclo[3.2.1]octan-6-yl hydrogen sulfate and a solvent, such that a solution of (2S,5R)-2-(5-(3-aminopropyl)-1,3,4-oxadiazol-2-yl)-7-oxo-1,6-diazabicyclo[3.2.1]octan-6-yl hydrogen sulfate is formed; and

(2) evaporating the solvent, such that Form 5 is formed.

In certain embodiments of these methods, the solvent is anisole, ethyl acetate, isopropyl acetate, methylisobutyl ketone, 2-propanol, dimethyl sulfoxide, t-butylmethyl ether, toluene, tetrahydrofuran, dichloromethane, acetonitrile, nitromethane, isopropyl alcohol, water, or mixtures thereof. In a particular embodiment, the solvent is water.

In step (1) of these methods, (2S,5R)-2-(5-(3-aminopropyl)-1,3,4-oxadiazol-2-yl)-7-oxo-1,6-diazabicyclo[3.2.1]octan-6-yl hydrogen sulfate can be in an amorphous form. In one embodiment, an amorphous form of the compound is formed by combining (2S,5R)-2-(5-(3-aminopropyl)-1,3,4-oxadiazol-2-yl)-7-oxo-1,6-diazabicyclo[3.2.1]octan-6-yl hydrogen sulfate with a solvent, such as water, followed by freeze drying or lyophilization.

In certain embodiments, any one of the above methods is a method of making Form 5 wherein the method further comprises: (3) drying the crystalline compound to obtain Form 5.

Also provided herein is a method of making Form 5 comprising:

(1) combining (2S,5R)-2-(5-(3-aminopropyl)-1,3,4-oxadiazol-2-yl)-7-oxo-1,6-diazabicyclo[3.2.1]octan-6-yl hydrogen sulfate and a solvent, such that a solution of (2S,5R)-2-(5-(3-aminopropyl)-1,3,4-oxadiazol-2-yl)-7-oxo-1,6-diazabicyclo[3.2.1]octan-6-yl hydrogen sulfate is formed; and

(2) combining an antisolvent with the solution, wherein the antisolvent is miscible with the solvent and wherein (2S,5R)-2-(5-(3-aminopropyl)-1,3,4-oxadiazol-2-yl)-7-oxo-1,6-diazabicyclo[3.2.1]octan-6-yl hydrogen sulfate is partially or completely insoluble in the antisolvent, such that crystalline Form 5 precipitates from the solution.

In certain embodiments of this solvent/antisolvent method, the solvent is water, and the antisolvent is THF or acetonitrile.

### Pharmaceutical Compositions

Provided herein are pharmaceutical compositions or formulations comprising Form 5 of the compound of Formula (I). Also provided herein are pharmaceutical compositions or formulations comprising Form 5 further comprising a β-lactam antibiotic.

The pharmaceutical compositions can be formulated for oral, intravenous, intramuscular, subcutaneous or parenteral administration for the therapeutic or prophylactic treatment of diseases, such as bacterial infections. Preferably, the pharmaceutical composition is formulated for intravenous administration.

The pharmaceutical preparations disclosed herein may be prepared in accordance with standard procedures and are administered at dosages that are selected to reduce, prevent or eliminate infection (see, e.g., Remington's Pharmaceutical Sciences, Mack Publishing Company, Easton, Pa. and Goodman and Gilman's “The Pharmaceutical Basis of Therapeutics,” Pergamon Press, New York, N.Y., the contents of which are incorporated herein by reference, for a general description of the methods for administering various antimicrobial agents for human therapy).

The pharmaceutical compositions can comprise one or more of the compounds disclosed herein, preferably Form 5 in conjunction with a β-lactam antibiotic, in association with one or more nontoxic, pharmaceutically-acceptable carriers and/or diluents and/or adjuvants and/or excipients. As used herein, the phrase “pharmaceutically-acceptable carrier” refers to any and all solvents, dispersion media, coatings, antibacterial and antifungal agents, isotonic and absorption delaying agents, and the like, that are compatible with pharmaceutical administration. The use of such media and agents for pharmaceutically active substances is well known in the art. Non-limiting examples of carriers and excipients include corn starch or gelatin, lactose, sucrose, microcrystalline cellulose, kaolin, mannitol, dicalcium phosphate, sodium chloride and alginic acid. The compositions may contain croscarmellose sodium, microcrystalline cellulose, corn starch, sodium starch glycolate and alginic acid.

Tablet binders that can be included are acacia, methylcellulose, sodium carboxymethylcellulose, polyvinylpyrrolidone (Povidone), hydroxypropyl methylcellulose, sucrose, starch and ethylcellulose.

Lubricants that can be used include magnesium stearate or other metallic stearates, stearic acid, silicone fluid, talc, waxes, oils and colloidal silica.

Flavoring agents such as peppermint, oil of wintergreen, cherry flavoring or the like can also be used. It may also be desirable to add a coloring agent to make the dosage form more aesthetic in appearance or to help identify the product.

For oral or parenteral administration, compounds of the present invention preferably in conjunction with a β-lactam antibiotic, can be mixed with conventional pharmaceutical carriers and excipients and used in the form of tablets, capsules, elixirs, suspensions, syrups, wafers and the like. The compositions comprising a compound of this invention may contain from about 0.1% to about 99% by weight of the active compound, such as from about 10% to about 30%.

For oral use, solid formulations such as tablets and capsules are useful. Sustained release or enterically coated preparations may also be devised. For pediatric and geriatric applications, one embodiment provides suspensions, syrups and chewable tablets. For oral administration, the pharmaceutical compositions are in the form of, for example, a tablet, capsule, suspension or liquid.

The pharmaceutical compositions may be made in the form of a dosage unit containing a therapeutically-effective amount of the active ingredient. Examples of such dosage units are tablets and capsules. For therapeutic purposes, the tablets and capsules which can contain, in addition to the active ingredient, conventional carriers such as binding agents, fillers, lubricants, disintegrants, or acceptable wetting agents. Oral liquid preparations generally are in the form of aqueous or oily solutions, suspensions, emulsions, syrups or elixirs.

For intravenous (IV) use, the pharmaceutical composition, preferably Form 5 in conjunction with a β-lactam antibiotic, can be dissolved or suspended in any of the commonly used intravenous fluids and administered by infusion. Intravenous fluids include, without limitation, physiological saline or Ringer's solution. Intravenous administration may be accomplished by using, without limitation, syringe, mini-pump or intravenous line.

Pharmaceutical compositions of this invention for parenteral injection comprise pharmaceutically-acceptable aqueous or non-aqueous solutions, dispersions, suspensions or emulsions as well as sterile powders for reconstitution into sterile injectable solutions or dispersions just prior to use.

Alternatively, the pharmaceutical compositions can be in powder form for reconstitution in the appropriate pharmaceutically acceptable carrier at the time of delivery. In another embodiment, the unit dosage form of compounds, preferably Form 5 in conjunction with a β-lactam antibiotic, can be a solution of one or more compounds, or salts thereof, in a suitable diluent, in sterile hermetically sealed ampoules or sterile syringes. The concentration of the compounds, preferably Form 5 in conjunction with a β-lactam antibiotic, in the unit dosage may vary, e.g. from about 1 percent to about 50 percent, depending on the compound used and its solubility and the dose desired by the physician. If the compositions contain dosage units, each dosage unit can contain from 1-500 mg of the active material. For adult human treatment, the dosage employed can range from 5 mg to 10 g, per day, depending on the route and frequency of administration.

The pharmaceutical compositions disclosed herein can be placed in a pharmaceutically acceptable carrier and are delivered to a recipient subject (e.g., a human) in accordance with known methods of drug delivery. In general, the methods of delivering the pharmaceutical compositions in vivo utilize art-recognized protocols for delivering the agent with the only substantial procedural modification being the substitution of the compounds of the present invention for the drugs in the art-recognized protocols. Likewise, methods for using the claimed compositions for treating cells in culture, for example, to eliminate or reduce the level of bacterial contamination of a cell culture, utilize art-recognized protocols for treating cell cultures with antibacterial agent(s) with the only substantial procedural modification being the substitution of the compounds of the present invention, preferably in combination with a β-lactam antibiotic for the drugs in the art-recognized protocols.

As used herein, the phrases “therapeutically-effective dose” and “therapeutically-effective amount” refer to an amount of a compound that prevents the onset, alleviates the symptoms, stops the progression of a bacterial infection, or results in another desired biological outcome such as, e.g., improved clinical signs or reduced/elevated levels of lymphocytes and/or antibodies. The term “treating” or “treatment” is defined as administering, to a subject, a therapeutically-effective amount of one or more compounds both to prevent the occurrence of an infection and to control or eliminate an infection. Those in need of treatment may include individuals already having a particular medical disease as well as those at risk for the disease (i.e., those who are likely to ultimately acquire the disorder). The term “subject,” as used herein, refers to a mammal, a plant, a lower animal, or a cell culture. In one embodiment, a subject is a human or other animal patient in need of antibacterial treatment.

The term “administering” or “administration” and the like, refers to providing the Form 5 to the subject in need of treatment. Preferably the subject is a mammal, more preferably a human. The present invention comprises administering Form 5 in conjunction with a β-lactam antibiotic. When Form 5 is administered in conjunction with a β-lactam antibiotic, Form 5 and the β-lactam antibiotic can be administered at the same time or different times. When Form 5 and the β-lactam antibiotic are administered at the same time, they can be administered as a single composition or pharmaceutical composition or they can be administered separately. It is understood that when Form 5 is administered in conjunction with a β-lactam antibiotic, that the active agents can be administered in a single combination or in multiple combinations. For example, when administered by IV, Form 5 can be dissolved or suspended in any of the commonly used intravenous fluids and administered by infusion, then a β-lactam antibiotic can be dissolved or suspended in any of the commonly used intravenous fluids and administered by infusion. Conversely the β-lactam antibiotic can be dissolved or suspended in any of the commonly used intravenous fluids and administered by infusion, then Form 5 can be dissolved or suspended in any of the commonly used intravenous fluids and administered by infusion. Alternatively, a pharmaceutical composition comprising Form 5 and a β-lactam antibiotic can be dissolved or suspended in any of the commonly used intravenous fluids and administered by infusion.

In one embodiment of the invention is provided a method of treating or preventing a bacterial infection comprising administering to a subject in need thereof a therapeutically-effective amount of the pharmaceutical composition comprising Form 5 and a β-lactam antibiotic.

In one embodiment of the invention is provided a method of treating or preventing a bacterial infection comprising administering to a subject in need thereof, a therapeutically-effective amount of a β-lactam antibiotic in conjunction with Form 5.

In one embodiment of the invention, is provided a method of treating or preventing a bacterial infection in a subject comprising the steps of
a. administering to the subject Form 5; then
b. administering a therapeutically-effective amount of a β-lactam antibiotic.

In one embodiment of the invention, is provided a method of treating or preventing a bacterial infection in a subject comprising the steps of
a. administering a therapeutically-effective amount of a β-lactam antibiotic; then
b. administering to the subject Form 5.

In one aspect, Form 5, preferably Form 5 in conjunction with a β-lactam antibiotic, can be used to treat a subject having a bacterial infection, wherein the infection is caused or exacerbated by any type of bacteria, such as Gram-negative bacteria. In one embodiment, the bacterial infection is caused by β-lactam resistant bacteria. In another embodiment, the bacterial infection is caused by β-lactamase producing bacteria. In another embodiment, the bacterial infection is caused by class A, class C, or class Dβ-lactamase producing bacteria. In another embodiment, the bacterial infection is caused by class A β-lactamase producing bacteria. In another embodiment, the infection is caused by class Cβ-lactamase producing bacteria. In still another embodiment, the infection is caused by class D β-lactamase producing bacteria. In still another embodiment, the infection is caused by KPC β-lactamase producing bacteria. In still another embodiment, the infection is caused by OXA β-lactamase producing bacteria.

Representative Gram-negative pathogens known to express β-lactamases include, but are not limited to Acinetobacter spp. (including Acinetobacter baumannii), Citrobacter spp., Escherichia spp. (including Escherichia coli), Haemophilus influenzae, Morganella morganii, Pseudomonas aeruginosa, Klebsiella spp. (including Klebsiella pneumoniae), Enterobacter spp. (including Enterobacter cloacae and Enterobacter aerogenes), Pasteurella spp., Proteus spp. (including Proteus Serratia spp. (including Serratia marcescens), and Providencia spp. Bacterial infections can be caused or exacerbated by Gram-negative bacteria including strains which express β-lactamases that may confer resistance to penicillins, cephalosporins, monobactams and/or carbapenems. The co-administration of a novel BLIs that inhibits these β-lactamases with a β-lactam antibiotic could be used to treat infections caused by β-lactam resistant bacteria.

In one aspect of the invention the infection is caused by a β-lactamase producing bacteria selected from Acinetobacter spp, Citrobacter spp, Escherichia coli, Enterobacter cloacae), Haemophilus influenzae, Pseudomonas aeruginosa, Proteus mirabilis, Serratia marcescens, and Klebsiella pneumoniae,

β-Lactam antibiotics that may be co-administered with Form 5 include, but are not limited to cephalosporin, carbapenem, monobactam, penem and penicillin classes of antibiotics.

In one embodiment of the invention, the β-lactam antibiotic is a cephalosporin. Examples of cephalosporins include, but are not limited to, Cefacetrile (cephacetrile), Cefadroxil (cefadroxyl), Cefalexin (cephalexin), Cefaloglycin (cephaloglycin), Cefalonium (cephalonium), Cefaloridine (cephaloradine), Cefalotin (cephalothin), Cefapirin (cephapirin), Cefatrizine, Cefazaflur, Cefazedone, Cefazolin (cephazolin), Cefradine (cephradine), Cefroxadine, Ceftezole, Cefaclor, Cefamandole, Cefmetazole, Cefonicid, Cefotetan, Cefoxitin, Cefprozil (cefproxil), Cefuroxime, Cefuzonam, Cefcapene, Cefdaloxime, Cefdinir, Cefditoren, Cefetamet, Cefixime, Cefmenoxime, Cefodizime, Cefotaxime, Cefpimizole, Cefpodoxime, Cefteram, Ceftibuten, Ceftiofur, Ceftiolene, Ceftizoxime, Ceftriaxone, Cefoperazone, Ceftazidime, Cefclidine, Cefepime, Cefluprenam, Cefoselis, Cefozopran, Cefpirome, Cefquinome, Cefaclomezine, Cefaloram, Cefaparole, Cefcanel, Cefedrolor, Cefempidone, Cefetrizole, Cefivitril, Cefmatilen, Cefmepidium, Cefovecin, Cefoxazole, Cefrotil, Cefsumide, Ceftaroline, Ceftioxide, Cefuracetime, cefbuperazone, cefminox, ceforanide, cefotiam, cefpiramide, cefsulodin, ceftobiprole latamoxef, loracarbef and Ceftolozane. In one embodiment the cephalosporin is Ceftolozane or Ceftazidime.

In one embodiment of the invention, the β-lactam antibiotic is a carbapenem. Examples of carbapenem antibiotics include, but are not limited to, Imipenem, Imipenem/Cilastatin, Biapenem, Doripenem, Meropenem, Ertapenem and Panipenem. In one embodiment the Carbapenem is Imipenem/Cilastatin or Meropenem.

In one embodiment of the invention, the β-lactam antibiotic is a monobactam. Examples of monobactam antibiotics include, but are not limited to Aztreonam, Tigemonam, Carumonam, BAL30072 and Nocardicin A.

In one embodiment of the invention, the β-lactam antibiotic is a penem. In one embodiment of the invention, the β-lactam antibiotic is a penicillin. Examples of penicillin antibiotics include, but are not limited to Amoxicillin, Ampicillin, Azlocillin, Mezlocillin, Apalcillin, Hetacillin, Becampicillin, Carbenicillin, Sulbenicillin, Ticarcillin, Piperacillin, Azlocillin, Mecillinam, Pivmecillinam, Methicillin, Ciclacillin, Talampicillin, Aspoxicillin, Oxacillin, Cloxacillin, Dicloxacillin, Flucloxacillin, Nafcillin and Pivampicillin.

The pharmaceutical compositions, preferably Form 5 in conjunction with a β-lactam antibiotic, can be used to treat a bacterial infection of any organ or tissue in the body caused by β-lactam resistant bacteria, preferably, Gram-negative β-lactam resistant bacteria. These organs or tissue include, without limitation, skeletal muscle, skin, bloodstream, kidneys, heart, lung and bone. For example, a pharmaceutical composition comprising at least Form 5, preferably Form 5 in conjunction with a β-lactam antibiotic, can be administered to a subject to treat, without limitation, skin and soft tissue infections (e.g., complex skin infections), bacteremia, intra-abdominal infections and urinary tract infections (e.g., cUTI). In addition, Form 5 may be used to treat community acquired respiratory infections, including, without limitation, otitis media, sinusitis, chronic bronchitis and pneumonia (including community-acquired pneumonia, hospital-acquired pneumonia and ventilator associated pneumonia), including pneumonia caused by drug-resistant Pseudomonas aeruginosa. Form 5, preferably Form 5 in conjunction with a β-lactam antibiotic, can be administered to a subject to treat mixed infections that comprise different types of Gram-negative bacteria, or which comprise both Gram-positive and Gram-negative bacteria. These types of infections include intra-abdominal infections and obstetrical/gynecological infections. Form 5, preferably in conjunction with a β-lactam antibiotic, may also be administered to a subject to treat an infection including, without limitation, endocarditis, nephritis, septic arthritis, intra-abdominal sepsis, bone and joint infections and osteomyelitis. Form 5, preferably in conjunction with a β-lactam antibiotic, or pharmaceutical compositions thereof, may also be directly injected or administered into an abscess, ventricle or joint. Pharmaceutical compositions administered as an aerosol for the treatment of pneumonia or other lung-based infections. In one embodiment, the aerosol delivery vehicle is an anhydrous, liquid or dry powder inhaler.

Actual dosage levels of active ingredients in the pharmaceutical compositions of Form 5, preferably Form 5 in conjunction with a β-lactam antibiotic, may be varied so as to obtain a therapeutically-effective amount of the active compound(s) to achieve the desired therapeutic response for a particular patient, compositions, and mode of administration. The effective amount can be determined as described herein. The selected dosage level will depend upon the activity of the particular compound, the route of administration, the severity of the condition being treated, and the condition and prior medical history of the patient being treated. However, it is within the skill of the art to start doses of the compound at levels lower than required to achieve the desired therapeutic effect and to gradually increase the dosage until the desired effect is achieved. In one embodiment, the data obtained from the assays can be used in formulating a range of dosage for use in humans. It will be understood by one of skill in the art that the when the composition comprises Form 5 and a β-lactam antibiotic, both Form 5 and the β-lactam antibiotic are active compounds.

Some embodiments of the method s described herein comprise administering to the subject an effective dose of Form 5, preferably in conjunction with a p lactam antibiotic. An effective dose of Form 5 is generally between 125 mg/day to 2000 mg/day. In one embodiment, an effective dose is from about 0.1 to about 100 mg/Kg of Form 5. In one embodiment, the dose is from about 0.1 to about 50 mg/Kg of Form 5. In another embodiment, the dose is from about 1 to about 25 mg/Kg of Form 5

Form 5, preferably Form 5 in conjunction with a β-lactam antibiotic, may be administered according to this method until the bacterial infection is eradicated or reduced. In one embodiment, Form 5, preferably a compound of Formula (I) in conjunction with a β-lactam antibiotic, are administered for a period of time from 3 days to 6 months. In another embodiment, Form 5, preferably Form 5 in conjunction with a β-lactam antibiotic, are administered for 7 to 56 days. In another embodiment, Form 5, preferably a compound of Form 5 in conjunction with a β-lactam antibiotic, are administered for 7 to 28 days. In a further embodiment, Form 5, preferably Form 5 in conjunction with a β-lactam antibiotic, are administered for 7 to 14 days.

Other embodiments provided herein include:

a pharmaceutical composition comprising Form 5 and at least one β-lactam antibiotic or a pharmaceutically acceptable salt thereof;

a pharmaceutical composition comprising Form 5 and at least one cephalosporin antibiotic or a pharmaceutically acceptable salt thereof;

a pharmaceutical composition comprising Form 5 and Ceftolozane antibiotic or a pharmaceutically acceptable salt thereof;

a pharmaceutical composition comprising Form 5 and at least one carbapenem antibiotic or a pharmaceutically acceptable salt thereof; and

a pharmaceutical composition comprising Form 5 and at least one monobactam antibiotic or a pharmaceutically acceptable salt thereof.

Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

### EXAMPLES

### Example I

### Synthesis of (2S,5R)-2-(5-(3-aminopropyl)-1,3,4-oxadiazol-2-yl)-7-oxo-1,6-diazabicyclo[3.2.1]octan-6-yl hydrogen sulfate (Formula (I))

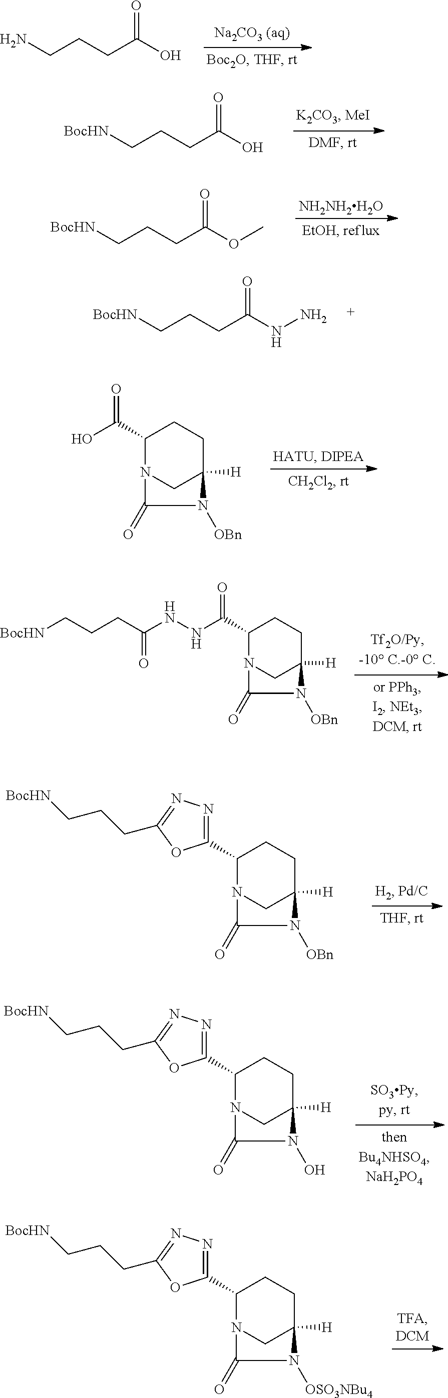

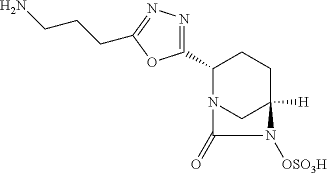

### Step 1: Synthesis of 4-(tert-butoxycarbonylamino)butanoic acid

To an aqueous solution of 4-aminobutanoic acid (25 g, 242 mmol) in H₂O (500 mL) at rt was added Na₂CO₃ (75 g, 726 mmol), followed by Boc₂O (95 g, 435 mmol) in THF (200 mL). The reaction mixture was stirred at room temperature (rt) for 12 hrs then concentrated under reduced pressure. The aqueous residue was extracted with Et₂O, then the aqueous layer was acidified to pH 4-5 with citric acid and extracted with EtOAc. The combined organic layer was dried over Na₂SO₄, and concentrated to afford 4-(tert-butoxycarbonylamino)butanoic acid (45 g, 90%) as a colorless oil. ESI-MS (EI⁺, m/z): 226 [M+Na]⁺.

### Step 2: Synthesis of methyl 4-(tert-butoxycarbonylamino)butanoate

To a solution of 4-(tert-butoxycarbonylamino)butanoic acid (7.0 g, 34.5 mmol) and K₂CO₃ (9.5 g, 68.9 mmol) in acetone (70 mL) was added MeI (7.5 g, 51.8 mmol) at rt. The reaction solution was stirred at 45° C. for 12 hrs. The mixture was washed with water and saturated sodium chloride, dried over Na₂SO₄, and concentrated to afford methyl 4-(tert-butoxycarbonylamino)-butanoate (6.2 g, 83%) as a yellow oil. ESI-MS (EI⁺, m/z): 240 [M+Na]⁺.

### Step 3: Synthesis of tert-butyl 4-hydrazinyl-4-oxobutylcarbamate

To a solution of methyl 4-(tert-butoxycarbonylamino)butanoate (21.0 g, 96.8 mmol) in MeOH (180 mL) was added NH₂NH₂.H₂O (28.0 g, 483 mmol) at rt. The mixture was stirred at 65° C. for 12 hrs then concentrated under reduced pressure. The crude material was dissolved in DCM (400 mL). The organic layer was washed with water (2×), and saturated sodium chloride (2×), dried over Na₂SO₄, and concentrated to afford tert-butyl 4-hydrazinyl-4-oxobutylcarbamate (18.9 g, 90%) as a white solid. ESI-MS (EI⁺, m/z): 240 [M+Na]⁺.

### Step 4: Synthesis of tert-butyl-4-(2-((2S,5R)-6-(benzyloxy)-7-oxo-1,6-diaza-bicyclo[3.2.1]octane-2-carbonyl)hydrazinyl)-4-oxobutylcarbamate

To a 0° C. solution of (2S,5R)-6-(benzyloxy)-7-oxo-1,6-diaza-bicyclo[3.2.1]octane-2-carboxylic acid (12.0 g, 43.5 mmol) and tert-butyl 4-hydrazinyl-4-oxobutylcarbamate (10.5 g, 47.8 mmol) in CH₂Cl₂ (360 mL) was added HATU (19.6 g, 52.2 mmol) and DIPEA (16.6 g, 130.5 mmol). The mixture was allowed to warm to rt, was stirred at rt for 12 hrs then diluted with CH₂Cl₂ (300 mL), washed with water (2×) and saturated sodium chloride (2×), dried over Na₂SO₄, and concentrated. The residue was purified by silica gel column chromatography (gradient elution 50-80% EtOAc/petroleum ether) to afford tert-butyl 4-(2-((2S,5R)-6-(benzyloxy)-7-oxo-1,6-diaza-bicyclo[3.2.1]octane-2-carbonyl)hydrazinyl)-4-oxobutylcarbamate (19.3 g, 93%) as a white solid. ESI-MS (EI⁺, m/z): 476 [M+H]⁺.

### Step 5: Synthesis of tert-butyl 3-(5-((2S,5R)-6-(benzyloxy)-7-oxo-1,6-diaza-bicyclo[3.2.1]octan-2-yl)-1,3,4-oxadiazol-2-yl)propylcarbamate

### Method A:

Tf₂O (8.0 mL, 0.0474 mol) was added dropwise to a −78° C. solution of tert-butyl 4-(2-((2S,5R)-6-(benzyloxy)-7-oxo-1,6-diaza-bicyclo[3.2.1]octane-2-carbonyl)hydrazinyl)-4-oxobutylcarbamate (7.5 g, 0.0158 mol) and Py (10.2 mL, 0.126 mol) in dry DCM (120 mL). The reaction mixture was allowed to warm to 0° C. then the reaction mixture was stirred at 0° C. for 3 hrs. Sat. NaHCO₃ was added at 0° C. very slowly. The organic layer was separated and the water layer was exacted with DCM (3×). The combined organic layer was washed with water, saturated sodium chloride, dried over Na₂SO₄, and concentrated. The residue was purified by silica gel column (gradient elution 0˜25% EtOAc/petroleum ether) to afford tert-butyl 3-(5-((2S,5R)-6-(benzyloxy)-7-oxo-1,6-diaza-bicyclo[3.2.1]octan-2-yl)-1,3,4-oxadiazol-2-yl)propylcarbamate (3.9 g, 54%) as a yellow solid. ESI-MS (EI⁺, m/z): 458 [M+H]⁺. ¹H NMR (500 MHz, CDCl₃) δ 7.45-7.37 (m, 5H), 5.08 (d, J=14.5 Hz, 1H), 4.93 (d, J=14.5 Hz, 1H), 4.70-4.66 (m, 1H), 3.37 (br s, 1H), 3.23-3.21 (m, 2H), 2.94-2.88 (m, 3H), 2.79 (d, J=14.5 Hz, 1H), 2.30-2.28 (m, 2H), 2.11-1.97 (m, 4H), 1.45 (s, 9H).

### Method B:

To a solution of PPh₃ (2.6 g, 10.0 mmol) in dry DCM (60 mL) was added I₂ (2.6 g, 10.0 mmol). After I₂ was dissolved completely, TEA (3.5 mL, 25.0 mmol) was added quickly at rt. The mixture was stirred for 15 mins. Tert-butyl 4-(2-((2S,5R)-6-(benzyloxy)-7-oxo-1,6-diaza-bicyclo[3.2.1]octane-2-carbonyl)hydrazinyl)-4-oxobutylcarbamate (2.4 g, 5.0 mmol) was added. The mixture was stirred at rt for 1 hr. The solvent was concentrated. EtOAc (250 mL) was added, and the solution was filtrated to remove POPh₃. The filtrate was concentrated. The resulting residue was purified by silica gel column chromatography (gradient elution 0-40% EtOAc/petroleum ether) to afford tert-butyl 3-(5-((2S,5R)-6-(benzyloxy)-7-oxo-1,6-diaza-bicyclo[3.2.1]octan-2-yl)-1,3,4-oxadiazol-2-yl)propylcarbamate (2.0 g, 86%) as a white solid. ESI-MS (EI⁺, m/z): 458 [M+H]⁺.

### Step 6-8

Following Steps 3-5 in Example 4, replacing tert-butyl (2-(5-((2S,5R)-6-(benzyloxy)-7-oxo-1,6-diazabicyclo[3.2.1]octan-2-yl)-1,3,4-oxadiazol-2-yl)ethyl)carbamate in Step 3 with tert-butyl (3-(5-((2S,5R)-6-(benzyloxy)-7-oxo-1,6-diazabicyclo[3.2.1]octan-2-yl)-1,3,4-oxadiazol-2-yl)propyl)carbamate; (2S,5R)-2-(5-(3-aminopropyl)-1,3,4-oxadiazol-2-yl)-7-oxo-1,6-diazabicyclo[3.2.1]octan-6-yl hydrogen sulfate (1.48 g) was obtained as a white solid after prep-HPLC purification using ammonium formate buffer. ESI-MS (EI⁺, m/z): 348.1. ¹H NMR (300 MHz, D₂O) δ 4.74 (d, J=6.2 Hz, 1H), 4.17 (br s, 1H), 3.17 (br d, J=12.1 Hz, 1H), 3.05-2.95 (m, 4H), 2.89 (d, J=12.3 Hz, 1H), 2.31-2.20 (m, 1H), 2.20-2.02 (m, 4H), 2.00-1.82 (m, 1H).

### Example 2

### Preparation of Amorphous Compound of Formula (I)

The amorphous form of the compound of Formula (I) can be prepared using the following procedures.

### Procedure 1—Freeze Drying

(2S,5R)-2-(5-(3-aminopropyl)-1,3,4-oxadiazol-2-yl)-7-oxo-1,6-diazabicyclo[3.2.1]octan-6-yl hydrogen sulfate (˜50 mg) was weighed into a 4 ml vial. Water (50 vol) was added and the sample heated to 50° C. for 30 minutes. The resulting solution was filtered and then rapidly frozen using dry ice and acetone. The frozen sample was placed on the freeze drier overnight. The resulting solid was analysed by XRPD.

### Procedure 2—Rapid Precipitation Using Anti-Solvent

(2S,5R)-2-(5-(3-aminopropyl)-1,3,4-oxadiazol-2-yl)-7-oxo-1,6-diazabicyclo[3.2.1]octan-6-yl hydrogen sulfate (˜20 mg) was weighed into 2 ml vials and dissolved in DMSO (4 vol) at room temperature. MIBK (25 vol) was then added and the samples were left at ambient conditions for 10 minutes. In all cases, gums formed. The solvent was then decanted and the residues were treated using the following procedures:

Procedure 2a—drying under ambient conditions for 30 min; or

Procedure 2b—drying in a vacuum oven at 50° C. for 2 h.

The samples were analysed by XRPD and, where appropriate, by DSC, TGA, VT-XRPD and ¹H NMR. Static stability at 25° C./96% RH and 40° C./75% RH was also investigated.

### Procedure 3—Heating to 120° C.

(2S,5R)-2-(5-(3-aminopropyl)-1,3,4-oxadiazol-2-yl)-7-oxo-1,6-diazabicyclo[3.2.1]octan-6-yl hydrogen sulfate (˜3.5 mg) was heated in a TGA pan to 120° C. at 10° C./min. The sample was then held at 120° C. for 10 minutes. The sample was analysed by XRPD and was found to be amorphous. ¹H NMR analysis did not show any signs of sample degradation.

This heating procedure was scaled up by heating (2S,5R)-2-(5-(3-aminopropyl)-1,3,4-oxadiazol-2-yl)-7-oxo-1,6-diazabicyclo[3.2.1]octan-6-yl hydrogen sulfate (˜800 mg) to 120° C. in an oven for 20 minutes. This material was analysed by XRPD TGA, GVS, and mDSC. Static stability experiments were carried out under ambient conditions and also at 25° C./96% RH and 40° C./75% RH. Finally this material was used as input material for the polymorphism screen (see Table 6).

### Example 3

### Polymorphism Studies

### Materials and Instruments

### 1. X-Ray Powder Diffraction:

Certain X-Ray Powder Diffraction patterns were collected on a Bruker AXS C2 GADDS diffractometer using Cu Kα radiation (40 kV, 40 mA), automated XYZ stage, laser video microscope for auto-sample positioning and a HiStar 2-dimensional area detector. X-ray optics consists of a single Gobel multilayer mirror coupled with a pinhole collimator of 0.3 mm. A weekly performance check is carried out using a certified standard NIST 1976 Corundum (flat plate).

The beam divergence, i.e. the effective size of the X-ray beam on the sample, was approximately 4 mm. A θ-θ continuous scan mode was employed with a sample—detector distance of 20 cm which gives an effective 20 range of 3.2°-29.7°. Typically the sample would be exposed to the X-ray beam for 120 seconds. The software used for data collection was GADDS for XP/2000 4.1.43 and the data were analysed and presented using Diffrac Plus EVA v13.0.0.2 or v15.0.0.0.

Samples run under ambient conditions were prepared as flat plate specimens using powder as received without grinding. Approximately 1-2 mg of the sample was lightly pressed on a glass slide to obtain a flat surface.

Samples run under non-ambient conditions were mounted on a silicon wafer with heat-conducting compound. The sample was then heated to the appropriate temperature at 20° C./min and subsequently held isothermally for 1 minute before data collection was initiated.

Other X-Ray Powder Diffraction patterns were collected on a Bruker D8 diffractometer using Cu Kα radiation (40 kV, 40 mA), 0-20 goniometer, and divergence of V4 and receiving slits, a Ge monochromator and a Lynxeye detector. The instrument is performance checked using a certified Corundum standard (NIST 1976). The software used for data collection was Diffrac Plus XRD Commander v2.6.1 and the data were analysed and presented using Diffrac Plus EVA v13.0.0.2 or v15.0.0.0.

Samples were run under ambient conditions as flat plate specimens using powder as received. The sample was gently packed into a cavity cut into polished, zero-background (510) silicon wafer. The sample was rotated in its own plane during analysis. The details of the data collection are: Angular range: 2 to 42° 2θ, Step size: 0.05° 2θ, Collection time: 0.5 s/step.

### 2. Single Crystal X-Ray Diffraction (SCXRD)

Data were collected on an Oxford Diffraction Supernova Dual Source, Cu at Zero, Atlas CCD diffractometer equipped with an Oxford Cryosystems Cobra cooling device. The data was collected using CuKα radiation. Structures were typically solved using either the SHELXS or SHELXD programs and refined with the SHELXL program as part of the Bruker AXS SHELXTL suite (V6.10). Unless otherwise stated, hydrogen atoms attached to carbon were placed geometrically and allowed to refine with a riding isotropic displacement parameter. Hydrogen atoms attached to a heteroatom were located in a difference Fourier synthesis and were allowed to refine freely with an isotropic displacement parameter.

### 3. Nuclear Magnetic Resonance (¹H-NMR)

NMR spectra were collected on a Bruker 400 MHz instrument equipped with an auto-sampler and controlled by a DRX400 console. Automated experiments were acquired using ICON-NMR v4.0.7 running with Topspin v1.3 using the standard Bruker loaded experiments. For non-routine spectroscopy, data were acquired through the use of Topspin alone. Samples were prepared in DMSO-d₆, unless otherwise stated. Off-line analysis was carried out using Topspin v1.3 or ACD SpecManager v12.5.

### 4. Differential Scanning Calorimetry (DSC)

Certain DSC data were collected on a TA Instruments Q2000 equipped with a 50 position auto-sampler. The calibration for thermal capacity was carried out using sapphire and the calibration for energy and temperature was carried out using certified indium. Typically 3-6 mg of sample was loaded into a pin-holed aluminium pan. A purge of dry nitrogen at 50 ml/min was maintained over the sample. Modulated temperature DSC was carried out using an underlying heating rate of 2° C./min and temperature modulation parameters of ±1.272° C. (amplitude) every 60 seconds (period). The instrument control software was Advantage for Q Series v2.8.0.394 and Thermal Advantage v5.2.6 and the data were analysed using Universal Analysis v4.7A or v4.4A. Other DSC data were collected on a Mettler DSC 823E equipped with a 34 position auto-sampler. The instrument was calibrated for energy and temperature using certified indium. Typically 0.5-3 mg of each sample, in a pin-holed aluminium pan, was heated at 10° C./min from 25° C. to 300° C. A nitrogen purge at 50 ml/min was maintained over the sample. The instrument control and data analysis software was STARe v9.20.

### 5. Thermo-Gravimetric Analysis (TGA)

TGA data were collected on a Mettler TGA/SDTA 851e equipped with a 34 position auto-sampler. The instrument was temperature calibrated using certified indium. Typically 3-10 mg of each sample was loaded onto a pre-weighed aluminium crucible and was heated at 10° C./min from ambient temperature to 350° C. A nitrogen purge at 50 ml/min was maintained over the sample. FIG. 3 depicts the differential scanning calorimetry (DSC) thermogram and thermogravimetry curve of Form 5.

### 6. Polarized Light Microscopy (PLM)

Samples were studied on a Leica LM/DM polarised light microscope with a digital video camera for image capture. A small amount of each sample was placed on a glass slide, mounted in immersion oil and covered with a glass slip, the individual particles being separated as well as possible. The sample was viewed with appropriate magnification and partially polarised light, coupled to a λ false-colour filter.

### 7. Water Determination by Karl Fischer Titration (KF)

The water content of each sample was measured on a Metrohm 874 Oven Sample Processor at 150° C. with 851 Titrano Coulometer using Hydranal Coulomat AG oven reagent and nitrogen purge. Weighed solid samples were introduced into a sealed sample vial. Approx 10 mg of sample was used per titration and duplicate determinations were made.

### 8. Chemical Purity Determination by HPLC

Purity analysis was performed on an Agilent HP1100 series system equipped with a diode array detector and using ChemStation software vB.02.01-SR1 (or SR2). Table 1 details the HPLC method parameters for chemical purity determination.

TABLE 1
HPLC Method Parameters for Chemical Purity Determinations

| Sample Preparation | 1 mg/ml in water | | |
|---|---|---|---|
| Column | 3.5 um Sunfire C18, 4.6 × 150 mm | | |
| Column Temperature (° C.) | 25 | | |
| Injection (μL) | 5 | | |
| Detection: | 215, 90 nm | | |
| Wavelength, Bandwidth (nm) | | | |
| Flow Rate (mL/min) | 2.0 | | |
| Phase A | 90:10 water:0.1% TFA in water | | |
| Phase B | 90:10 acetonitrile:0.1% TFA in water | | |
| Timetable | Time (min) | % Phase A | % Phase B |
| | 0.00 | 90 | 10 |
| | 2.00 | 90 | 10 |
| | 5.00 | 60 | 40 |
| | 8.00 | 0 | 100 |
| | 8.01 | 90 | 10 |
| | 10.00 | 90 | 10 |

### 9. Gravimetric Vapour Sorption (GVS)

Sorption isotherms were obtained using a SMS DVS Intrinsic moisture sorption analyser, controlled by DVS Intrinsic Control software v1.0.1.2 (or v 1.0.1.3). The sample temperature was maintained at 25° C. by the instrument controls. The humidity was controlled by mixing streams of dry and wet nitrogen, with a total flow rate of 200 ml/min The relative humidity was measured by a calibrated Rotronic probe (dynamic range of 1.0-100% RH), located near the sample. The weight change, (mass relaxation) of the sample as a function of % RH was constantly monitored by the microbalance (accuracy±0.005 mg). Typically 5-20 mg of sample was placed in a tared mesh stainless steel basket under ambient conditions. The sample was loaded and unloaded at 40% RH and 25° C. (typical room conditions). A moisture sorption isotherm was performed as outlined below (2 scans giving 1 complete cycle or 4 scans giving 2 complete cycles). The standard isotherm was performed at 25° C. at 10% RH intervals over a 0-90% RH range. Data analysis was undertaken in Microsoft Excel using DVS Analysis Suite v6.2 (or 6.1 or 6.0). Table 2 details the method parameters for SMS DVS intrinsic experiments.

TABLE 2
Method Parameters for SMS DVS Intrinsic Experiments

| Parameters | Values |
|---|---|
| Adsorption - Scans 1 and 3 | 40-90 |
| Desorption/Adsorption - Scans 2 and 4 | 90-0, 0-40 |
| Intervals (% RH) | 10 |
| Number of Scans | 2 or 4 |
| Flow rate (ml/min) | 200 |
| Temperature (° C.) | 25 |
| Stability (° C./min) | 0.2 |
| Sorption Time (hours) | 6 hour time out |

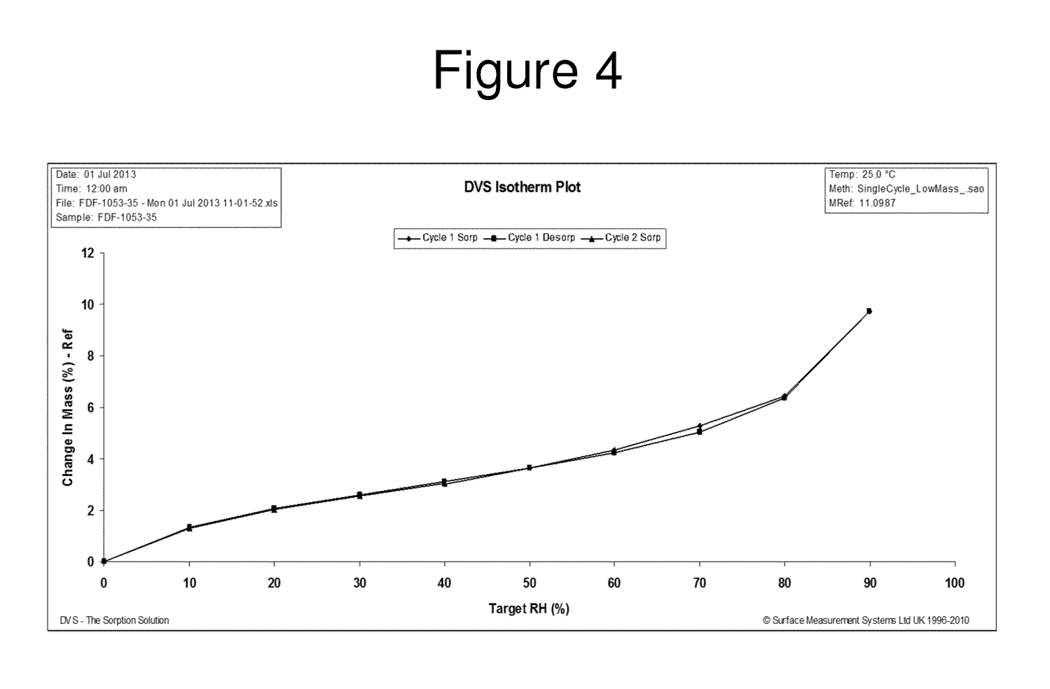

The sample was recovered after completion of the isotherm and re-analysed by XRPD. FIG. 4 depicts the DVS graph of Form 5 of (2S,5R)-2-(5-(3-aminopropyl)-1,3,4-oxadiazol-2-yl)-7-oxo-1,6-diazabicyclo[3.2.1]octan-6-yl hydrogen sulfate.

### Results

The following polymorphism studies carried out on (2S,5R)-2-(5-(3-aminopropyl)-1,3,4-oxadiazol-2-yl)-7-oxo-1,6-diazabicyclo[3.2.1]octan-6-yl hydrogen sulfate. A number of different conditions, including a diverse range of solvent systems and temperature profiles, were used during this investigation. In summary, nine different crystalline solid forms were identified for the compound of Formula (I).

Solid state characterization was performed on five of the seven forms, together with an assessment of stability relationships between the different hydrates. The remaining forms could not be isolated due to their metastable nature.

Based on these findings, it was concluded that Form 5 is the most stable crystalline form at ambient conditions.

### 1. Screen Procedure and Results

A wide range of methodologies was used in an attempt to fully evaluate the polymorphic landscape of the sodium salt. These include traditional crystallization, slow evaporation, heat/cool cycles and suspension/equilibration techniques. Slurry ripening or slurry maturation increases the possibility of generating metastable forms in accordance with the Ostwald rule of stages (Ostwald, W. (1897). “Studien über die Bildung und Umwandlung fester Körper. 1. Abhandlung: Übersättigung und Überkaltung”. Zeitschrift für Physikalische Chemie 22: 289-330).

### Solubility Screen Using Crystalline Material

(2S,5R)-2-(5-(3-aminopropyl)-1,3,4-oxadiazol-2-yl)-7-oxo-1,6-diazabicyclo[3.2.1]octan-6-yl hydrogen sulfate (˜30 mg) was weighed into 4 ml vials and treated with increasing amounts of solvent. The solvent was added in increments until the solid dissolved or a maximum of 100 volumes had been reached. In between each addition of solvent the samples were shaken at 50° C. for ˜15 minutes.

Slurries were matured (shaken in cycles of 4 hours at R.T./4 hours at 50° C.). After 3 days of maturation, the samples were cooled down to room temperature and small amounts of each solid obtained were pipetted onto glass slides for analysis by XRPD. Samples which gave new diffractograms were filtered for further characterisation.

The solutions were split into two and one of the following procedures was followed:

Procedure 1a—evaporation under ambient conditions

Procedure 1b—cooling to 4° C. at 0.1° C./min

Table 3 shows the results of the initial screen where the crystalline forms were identified and denoted Forms 1, 2, 3, 4, 5, 6, 7, 8, or 9.

TABLE 3
Results from Polymorphism Screen (Procedures 1a and 1b)

Crystalline starting material

| Solvent | 1 | 1a | 1b |
|---|---|---|---|
| n-Heptane | Form 5 | N/A | N/A |
| Diethyl ether | Form 5 | N/A | N/A |
| Cumene | Form 5 | N/A | N/A |
| Ethyl acetate | Form 4 | N/A | N/A |
| Isopropyl acetate | Form 4 | N/A | N/A |
| MIBK | Form 5 | N/A | N/A |
| 2-Propanol | Form 5 | N/A | N/A |
| MEK | Form 7 | N/A | N/A |
| Acetone | mostly | N/A | N/A |
| | amorphous | | |
| TBME | Pattern 2 | N/A | N/A |
| Toluene | Form 5 | N/A | N/A |
| DIPE | Pattern 2 | N/A | N/A |
| | (new form + | | |
| | Form 5) | | |
| 1,2-Dimethoxyethane | Form 4 | N/A | N/A |
| THF | Form 4 | N/A | N/A |
| DCM | Form 5 | N/A | N/A |
| DMF | N/A | no solid | Form 5 |
| | | obtained | |
| Acetonitrile | Form 5 | N/A | N/A |
| Nitromethane | Form 6 | N/A | N/A |
| Ethylene glycol | N/A | Form 5 | no solid |
| | | | obtained |
| Water | N/A | Form 5 | Form 5 |
| Water:THF (2:98) | Form 5 | N/A | N/A |
| Water:IPA (2:98) | Form 5 | N/A | N/A |
| Water:Acetone (2:98) | Form 5 | N/A | N/A |
| Water:THF (5:95) | Form 5 | N/A | N/A |
| Water:IPA (5:95) | Form 5 | N/A | N/A |
| Water:Acetone (5:95) | Form 5 | N/A | N/A |

### Solubility Screen Using Amorphous Material

The same solvent set used for the solubility screen was used for the polymorphism screen starting from amorphous material, with the exception of those solvents in which the crystalline material fully dissolved.

Amorphous (2S,5R)-2-(5-(3-aminopropyl)-1,3,4-oxadiazol-2-yl)-7-oxo-1,6-diazabicyclo[3.2.1]octan-6-yl hydrogen sulfate (˜30 mg) was weighed into 4 ml vials and treated with increasing amounts of solvent. The solvent was added in increments until the solid dissolved or a maximum of 100 volumes had been used. Between each addition of solvent, the samples were shaken at 50° C. for ˜15 minutes.

Slurries were split into three 2 ml vials and treated using the following procedures:

Procedure 2a

maturation between R.T and 50° C. (4 hours at each temperature)

Procedure 2b

slurrying at 4° C.

Procedure 2c

slurrying at 50° C.

After 24 h, the samples were equilibrated to room temperature and small amounts of each were pipetted onto glass slides for XRPD analysis.

Table 4 shows the results of the initial screen where the crystalline forms were identified and denoted Forms 1, 2, 3, 4, 5, 6, 7, 8, or 9.

TABLE 4
Results from Polymorphism Screen (Procedures 2a, 2b, and 2c)

| | Amorphous starting material | | |
|---|---|---|---|
| Solvent | 2a | 2b | 2c |
| n-Heptane | Amorphous | Amorphous + | Amorphous |
| | | Form 5 | |
| Diethyl ether | Amorphous | Form 5 | Form 5 |
| Cumene | Amorphous | Amorphous + | Amorphous |
| | | Form 5 | |
| Ethyl acetate | Form 5 | Form 5 | Form 5 |
| Isopropyl acetate | Form 5 | Form 5 | Form 5 |
| MIBK | Amorphous | Form 5 | Amorphous |
| 2-Propanol | Form 5 | Form 5 | Form 5 |
| MEK | Form 5 | Form 5 | Form 5 |
| Acetone | Form 5 | Form 5 | Form 5 |
| TBME | Form 5 | Form 5 | Form 5 |
| Toluene | Amorphous | Form 5 | Amorphous |
| DIPE | Form 5 | Form 5 | Form 5 |
| 1,2-Dimethoxyethane | Form 5 | Form 5 | Form 5 + |
| | | | Form 4 |
| THF | Form 5 | Form 5 | Form 5 |
| DCM | Form 5 | Form 5 | Form 5 |
| DMF | N/A | N/A | N/A |
| Acetonitrile | Form 5 | Form 5 | Form 5 |
| Nitromethane | Form 6 | Form 5 | Form 5 + |
| | | | Form 6 |
| Ethylene glycol | N/A | N/A | N/A |
| Water | N/A | N/A | N/A |
| Water:THF (2:98) | Form 5 | Form 5 | Form 5 |
| Water:IPA (2:98) | Form 5 | Form 5 | Form 5 |
| Water:Acetone (2:98) | Form 5 | Form 5 | Form 5 |
| Water:THF (5:95) | Form 5 | Form 5 | Form 5 |
| Water:IPA (5:95) | Form 5 | Form 5 | Form 5 |
| Water:Acetone (5:95) | Form 5 | Form 5 | Form 5 |

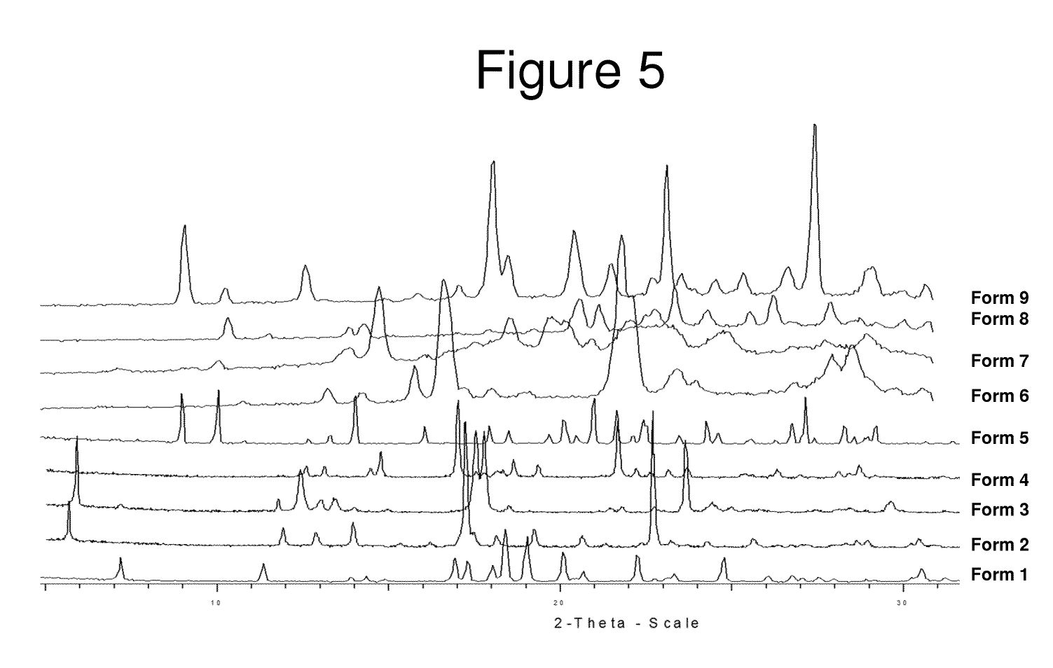

FIG. 5 shows XRPD spectra of the various polymorph forms of (2S,5R)-2-(5-(3-aminopropyl)-1,3,4-oxadiazol-2-yl)-7-oxo-1,6-diazabicyclo[3.2.1]octan-6-yl hydrogen sulfate.

Form 5 is the most stable crystalline form of (2S,5R)-2-(5-(3-aminopropyl)-1,3,4-oxadiazol-2-yl)-7-oxo-1,6-diazabicyclo[3.2.1]octan-6-yl hydrogen sulfate at ambient conditions (25° C. and 40% RH) (see FIG. 2) compared to the other hydrates described herein.

The diagram on FIG. 6 summarizes the relative thermodynamic relationships between the different crystalline solid forms of (2S,5R)-2-(5-(3-aminopropyl)-1,3,4-oxadiazol-2-yl)-7-oxo-1,6-diazabicyclo[3.2.1]octan-6-yl hydrogen sulfate.

The solid forms of (2S,5R)-2-(5-(3-aminopropyl)-1,3,4-oxadiazol-2-yl)-7-oxo-1,6-diazabicyclo[3.2.1]octan-6-yl hydrogen sulfate were further characterized using a variety of techniques. The results are summarised in Tables 5-10.

TABLE 5
Characterization of Amorphous Solid

| Technique | Result |
|---|---|
| XRPD | Amorphous |
| ¹H NMR | Consistent with structure, no signs of degradation |
| mDSC | Tg at ~97° C. |
| Static Stability - | Form 5 peaks observed after 36 days (although |
| ambient | peaks are small) |
| Static Stability - | Conversion to Form 5 within 15 minutes |
| 25° C./96% RH | |
| Static Stability - | Conversion to Form 5 within 15 minutes |
| 40° C./75% RH | |
| TGA | Weight loss of 4.9% from onset to 120° C. |
| GVS | Rapid uptake of water from 40-60% |
| | RH then more gradual. |
| | Sample contains more water at the end of the |
| | analysis than at the start, suggesting |
| | hydration. This was confirmed by |
| | XRPD analysis of resulting the solid (Form 5) |
| VT-XRPD | Material did not crystallise on heating |

TABLE 6
Summary of Results for Scale Up of Form 5

| Technique | | Result |
|---|---|---|
| High Resolution XRPD | | Form 5 |
| ¹H NMR | | Consistent with structure. No |
| | | residual ammonium formate |
| TGA | | 4.99% weight loss from 25° C. |
| | | to 125° C., |
| | | 5.46% weight loss from 125-145° C. |
| DSC | | Weight loss endotherm with onset of |
| | | 125° C. |
| Microscopy | | Needles ranging from ~130 μm |
| | | up to ~900 μm. Also some very |
| | | small particles ~30 μm. |
| GVS | | Mass change of 9.73% from 0-90% RH |
| XRPD post GVS | | Form 5. No change in crystallinity |
| HPLC | Sample 1 | 90.8% |
| | Sample 2 | 99.2% |

| | |
|---|---|
| KF | 10.70% (2.3 moles) |
| Aqueous solubility | 13.1 mg/ml (pH 4.85). |
| (24 hrs at 25°) | Residue was Form 5 |
| Storage at 25° C./ | No change in form or crystallinity |
| 96% RH for 1 week | |
| Storage at 40° C./ | No change in form or crystallinity |
| 75% RH for 1 week | |
| Storage in vacuum | No change in form or crystallinity |
| oven at 25° C. | after 5 days |
| Storage in vacuum | Sample almost completely amorphous |
| oven at 50° C. | after 24 h |
| XRPD after ball milling | No change in form but loss of |
| (20 min) | crystallinity |

TABLE 7
Summary of Characterisation on Forms 6, 7 and 9

| Analysis/Test | Form 6 | Form 7 | Form 9 |
|---|---|---|---|
| Drying in at 5 | Partial conversion to | Partial conversion to | No change in form |
| mmHg/25° C. | Form 5 | Form 5 | |
| overnight | | | |
| Filtration and air | No change in form | No change in form | N/A |
| drying for 30 min | | | |
| Storage at ambient | Partial conversion to | No change in form | No change in form |
| conditions for 24 h | Form 5 | | |
| TGA | 13.8% weight loss | Weight loss of 4.1% | Weight loss of 3.19 from |
| | from 25° C. to | from 25° C. to 150° C. | 25° C. to 140° C. 13.62% |
| | degradation | | weight loss from 140-180° C. |
| | (~180° C.) | | |
| DSC | Weight loss | No events before | Endotherm just before |
| | endotherm from 65- | degradation | degradation; onset |
| | 105° C. | | ~154° C. |
| ¹H NMR | Consistent with | Consistent with | Consistent with structure. |
| | structure. ~0.7 molar | structure. ~0.15 | ~1 molar eq. residual |
| | eq. residual | molar eq. residual | DMSO and ~0.03 molar |
| | nitromethane | MEK | eq. residual MIBK |
| Storage at at | Conversion to Form 5 | Conversion to Form 5 | Conversion to Form 5 |
| 25° C./96% RH | | | |
| for 24 h | | | |
| Storage at at | Conversion to Form 5 | Conversion to Form 5 | Conversion to Form 5 |
| 40° C./75% RH | | | |
| for 24 h | | | |
| HPLC | N/A | 93.0% | N/A |
| KF | N/A | 4.09% (suspected | N/A |
| | | surface bound | |
| | | water) | |
| VT-XRPD | N/A | No change in form | Amorphous by 175° C. |
| | | before degradation | |

TABLE 8
Summary of Forms

| Form | Nature of Form |
|---|---|
| 1 | Suspected non-solvated form |
| 2 | Suspected non-solvated form |
| 3 | Suspected non-solvated form |
| 4 | Suspected non-solvated form |
| 5 | 2.7 hydrate (crystal structure obtained) |
| 6 | solvation state unknown |
| 7 | Suspected non-solvated form |
| 8 | solvation state unknown |
| 9 | solvation state unknown |

TABLE 9
XRPD Scanning Data of Form 5 (FIG. 1)

| 2-Theta Angle | Intensity % |
|---|---|
| 9.013 | 29.5 |
| 9.993 | 100.0 |
| 10.809 | 4.1 |
| 12.703 | 10.2 |
| 13.291 | 8.4 |
| 14.010 | 28.8 |
| 16.034 | 15.7 |
| 16.720 | 4.4 |
| 17.928 | 22.9 |
| 18.483 | 21.6 |
| 19.724 | 9.4 |
| 20.149 | 37.9 |
| 20.508 | 7.6 |
| 21.031 | 42.9 |
| 21.651 | 13.8 |
| 22.141 | 7.1 |
| 22.533 | 49.4 |
| 23.480 | 6.5 |
| 24.329 | 25.3 |
| 24.623 | 14.7 |
| 25.537 | 9.4 |
| 26.288 | 4.5 |
| 26.778 | 14.1 |
| 27.170 | 20.3 |
| 27.431 | 5.5 |
| 28.280 | 13.1 |
| 28.574 | 9.6 |
| 28.966 | 12.3 |
| 29.260 | 26.9 |
| 30.697 | 4.2 |
| 31.481 | 3.2 |
| 31.971 | 5.8 |
| 32.460 | 4.4 |
| 32.722 | 4.9 |
| 33.113 | 5.0 |
| 33.767 | 4.4 |
| 34.224 | 7.1 |
| 34.975 | 4.7 |
| 35.269 | 3.7 |
| 36.151 | 4.4 |
| 36.771 | 17.5 |
| 37.000 | 5.8 |
| 37.718 | 9.7 |
| 37.914 | 10.5 |
| 38.143 | 4.7 |
| 38.926 | 4.7 |
| 41.082 | 3.2 |
| 41.735 | 2.9 |

TABLE 10
XRPD Scanning Data of Forms 1, 2, 3, 4, 5, 6, 7, 8, 9 (FIG. 5)

| | |
|---|---|
| Angle | Intensity % |
| 2Θ ° | % |

Form 1

| | |
|---|---|
| 7.183 | 48.7 |
| 11.363 | 36.4 |
| 13.877 | 12.7 |
| 14.334 | 13.1 |
| 14.922 | 7.3 |
| 16.979 | 46.8 |
| 17.338 | 42.2 |
| 18.057 | 33.4 |
| 18.416 | 100.0 |
| 19.069 | 86.6 |
| 20.114 | 58.4 |
| 20.701 | 22.8 |
| 22.269 | 53.2 |
| 22.791 | 8.6 |
| 23.379 | 18.3 |
| 23.967 | 5.4 |
| 24.783 | 48.3 |
| 25.273 | 5.6 |
| 26.089 | 13.8 |
| 26.775 | 15.1 |
| 27.101 | 9.7 |
| 27.558 | 12.9 |
| 28.015 | 9.1 |
| 28.930 | 7.1 |
| 29.126 | 7.5 |
| 30.562 | 27.6 |
| 30.954 | 7.3 |
| 31.248 | 10.8 |
| 31.640 | 6.5 |
| 32.391 | 10.6 |
| 33.762 | 5.8 |
| 34.285 | 7.5 |
| 35.036 | 8.2 |
| 35.493 | 5.8 |
| 35.721 | 5.6 |
| 36.440 | 7.1 |
| 36.995 | 7.1 |
| 37.223 | 8.2 |
| 37.550 | 11.2 |
| 39.280 | 7.3 |
| 40.162 | 11.2 |

Form 2

| | |
|---|---|
| 5.655 | 36.4 |
| 11.939 | 16.6 |
| 12.881 | 12.8 |
| 13.938 | 20.4 |
| 15.338 | 5.2 |
| 16.195 | 6.2 |
| 17.280 | 94.5 |
| 17.508 | 12.9 |
| 18.165 | 10.2 |
| 19.251 | 15.2 |
| 20.650 | 10.2 |
| 21.336 | 5.2 |
| 22.393 | 5.0 |
| 22.735 | 100.0 |
| 23.249 | 7.2 |
| 23.706 | 4.1 |
| 24.335 | 5.9 |
| 25.649 | 8.7 |
| 28.676 | 6.5 |
| 28.990 | 6.8 |
| 30.276 | 4.7 |
| 30.504 | 8.4 |
| 30.961 | 4.0 |

Form 3

| | |
|---|---|
| 5.912 | 95.2 |
| 7.169 | 13.2 |
| 11.796 | 19.8 |
| 12.453 | 54.1 |
| 13.024 | 18.0 |
| 13.481 | 20.8 |
| 14.024 | 9.2 |
| 14.995 | 7.9 |
| 17.566 | 100.0 |
| 17.794 | 98.2 |
| 18.508 | 11.4 |
| 21.164 | 6.1 |
| 21.450 | 8.8 |
| 21.821 | 10.6 |
| 22.764 | 10.2 |
| 23.649 | 89.7 |
| 24.420 | 15.5 |
| 25.020 | 9.8 |
| 26.648 | 5.0 |
| 27.505 | 6.2 |
| 28.133 | 8.0 |
| 28.505 | 8.0 |
| 28.905 | 6.2 |
| 29.647 | 17.1 |
| 31.218 | 6.2 |
| 32.018 | 5.9 |
| 32.389 | 5.6 |
| 32.846 | 5.0 |
| 33.532 | 4.8 |
| 34.674 | 6.2 |
| 35.845 | 5.3 |
| 36.673 | 5.5 |
| 37.359 | 5.2 |

Form 4

| | |
|---|---|
| 12.596 | 18.8 |
| 13.110 | 18.8 |
| 14.481 | 16.3 |
| 14.766 | 37.5 |
| 17.051 | 100.0 |
| 17.537 | 12.2 |
| 17.823 | 10.0 |
| 18.194 | 13.1 |
| 18.308 | 13.2 |
| 18.622 | 27.1 |
| 19.365 | 20.0 |
| 21.678 | 76.3 |
| 22.221 | 15.8 |
| 22.650 | 11.1 |
| 23.164 | 14.0 |
| 23.735 | 18.8 |
| 25.420 | 9.4 |
| 25.620 | 8.3 |
| 26.334 | 15.1 |
| 27.020 | 6.9 |
| 27.362 | 5.2 |
| 28.133 | 12.2 |
| 28.448 | 10.0 |
| 28.762 | 20.6 |
| 29.190 | 6.8 |
| 31.190 | 6.3 |
| 31.932 | 6.3 |
| 34.389 | 8.0 |
| 34.874 | 8.8 |
| 35.160 | 7.4 |
| 36.759 | 5.8 |
| 38.387 | 4.9 |
| 39.444 | 5.5 |

Form 5

| | |
|---|---|
| 9.013 | 29.5 |
| 9.993 | 100.0 |
| 10.809 | 4.1 |
| 12.703 | 10.2 |
| 13.291 | 8.4 |
| 14.010 | 28.8 |
| 16.034 | 15.7 |
| 16.720 | 4.4 |
| 17.928 | 22.9 |
| 18.483 | 21.6 |
| 19.724 | 9.4 |
| 20.149 | 37.9 |
| 20.508 | 7.6 |
| 21.031 | 42.9 |
| 21.651 | 13.8 |
| 22.141 | 7.1 |
| 22.533 | 49.4 |
| 23.480 | 6.5 |
| 24.329 | 25.3 |
| 24.623 | 14.7 |
| 25.537 | 9.4 |
| 26.288 | 4.5 |
| 26.778 | 14.1 |
| 27.170 | 20.3 |
| 27.431 | 5.5 |
| 28.280 | 13.1 |
| 28.574 | 9.6 |
| 28.966 | 12.3 |
| 29.260 | 26.9 |
| 30.697 | 4.2 |
| 31.481 | 3.2 |
| 31.971 | 5.8 |
| 32.460 | 4.4 |
| 32.722 | 4.9 |
| 33.113 | 5.0 |
| 33.767 | 4.4 |
| 34.224 | 7.1 |
| 34.975 | 4.7 |
| 35.269 | 3.7 |
| 36.151 | 4.4 |
| 36.771 | 17.5 |
| 37.000 | 5.8 |
| 37.718 | 9.7 |
| 37.914 | 10.5 |
| 38.143 | 4.7 |
| 38.926 | 4.7 |
| 41.082 | 3.2 |
| 41.735 | 2.9 |

Form 6

| | |
|---|---|
| 10.731 | 6.7 |
| 13.203 | 14.3 |
| 14.205 | 11.2 |
| 15.741 | 26.9 |
| 16.587 | 75.0 |
| 17.233 | 14.6 |
| 18.012 | 13.9 |
| 21.797 | 100.0 |
| 22.109 | 66.5 |
| 23.445 | 25.9 |
| 23.957 | 18.4 |
| 26.161 | 13.2 |
| 26.829 | 17.0 |
| 27.118 | 15.7 |
| 27.497 | 20.1 |
| 27.942 | 34.0 |
| 28.521 | 38.2 |
| 29.790 | 14.5 |
| 30.569 | 14.3 |

Form 7

| | |
|---|---|
| 7.078 | 11.0 |
| 9.014 | 10.7 |
| 9.326 | 11.1 |
| 10.038 | 19.4 |
| 13.844 | 33.3 |
| 14.734 | 100.0 |
| 16.047 | 25.0 |
| 16.181 | 25.2 |
| 17.516 | 40.1 |
| 18.562 | 65.4 |
| 19.764 | 67.5 |
| 20.098 | 63.3 |
| 20.943 | 42.6 |
| 22.079 | 63.7 |
| 22.524 | 69.7 |
| 22.724 | 75.8 |
| 23.325 | 58.3 |
| 24.772 | 52.1 |
| 26.752 | 34.9 |
| 27.198 | 35.1 |
| 27.754 | 42.2 |
| 28.934 | 48.6 |

Form 8

| | |
|---|---|
| 10.305 | 46.8 |
| 11.507 | 18.2 |
| 13.799 | 28.4 |
| 14.289 | 35.2 |
| 14.934 | 14.8 |
| 15.157 | 14.5 |
| 17.004 | 18.3 |
| 17.961 | 23.7 |
| 19.274 | 25.8 |
| 20.565 | 79.8 |
| 21.144 | 69.3 |
| 23.369 | 100.0 |
| 24.304 | 59.9 |
| 25.551 | 58.3 |
| 26.218 | 87.6 |
| 26.708 | 39.4 |
| 27.910 | 74.8 |
| 28.622 | 41.6 |
| 28.778 | 46.0 |
| 29.045 | 35.2 |
| 29.223 | 37.3 |
| 30.069 | 43.3 |
| 30.759 | 39.8 |

Form 9

| | |
|---|---|
| 9.014 | 21.6 |
| 10.216 | 10.8 |
| 12.152 | 36.4 |
| 12.530 | 36.8 |
| 13.109 | 18.4 |
| 15.913 | 25.6 |
| 16.982 | 29.6 |
| 18.050 | 80.4 |
| 18.473 | 100.0 |
| 19.274 | 33.6 |
| 19.986 | 40.0 |
| 20.409 | 42.8 |
| 21.500 | 72.8 |
| 22.680 | 61.6 |
| 23.125 | 47.6 |
| 23.548 | 38.8 |
| 24.505 | 46.0 |
| 25.306 | 35.2 |
| 25.884 | 51.2 |
| 26.708 | 87.2 |
| 27.376 | 45.6 |
| 29.112 | 40.4 |
| 30.024 | 31.6 |

### Example 4

### Single Crystal Experiments

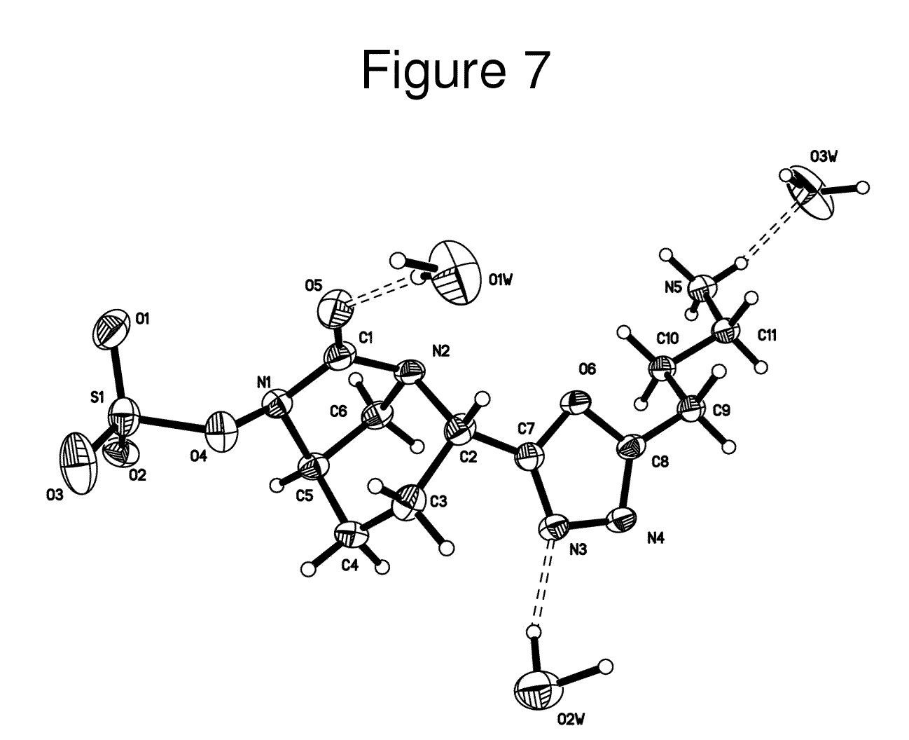

Single crystals of Form 5 were grown by slow evaporation (at ambient conditions) from a solution in water. This sample was submitted for single crystal X-ray diffraction studies and the structure was obtained. The results are shown in Table 11 and FIG. 7.

TABLE 11
Single Crystal Structure of Form 5

| Molecular formula | C₁₁H₂₃N₅O₉S | | | | |
|---|---|---|---|---|---|
| Molecular formula | 401.4 | | | | |
| Molecular weight | Orthorhombic | | | | |
| Crystal system | P2₁2₁2₁ | a | 8.8951(2) Å, | α | 90°, |
| Space group | | b | 9.7461(2) Å, | β | 90°, |
| | | c | 19.7741(4) Å, | γ | 90° |

| | |
|---|---|
| | 1714.27(6) Å³ |
| V | 4 |
| Z | 1.555 g · cm⁻³ |
| D_{c} | 2.234 mm⁻¹ |
| μ | Cu—K(alpha), 1.54178 Å |
| Source, λ | Cu—K(alpha), 1.54178 Å |
| F(000) | 848 |
| T | 100(2)K |
| Crystal | Colorless prism, 0.85 × 0.25 × 0.15 mm |
| Data truncated to | 0.80 Å |
| θₘₐₓ | 74.44° |
| Completeness | 99.5% |
| Reflections | 33296 |
| Unique reflections | 3494 |
| Rᵢₙₜ | 0.0611 |

The structure solution was obtained by direct methods, full-matrix least-squares refinement on F² with weighting w⁻¹=σ²(Fₒ²)+(0.0800P)²+(1.3600P), where P=(Fₒ²+2F_{c}²)/3, anisotropic displacement parameters, empirical absorption correction using spherical harmonics, implemented in SCALE3 ABSPACK scaling algorithm, absolute structure parameter=0.02(2). Final wR²={Σ[w(Fₒ²−F_{c}²)²]/Σ[w(Fₒ²)²]^{1/2}}=0.1242 for all data, conventional R₁=0.0452 on F values of 3474 reflections with Fₒ>4σ(Fₒ), S=1 for all data and 255 parameters. Final Δ/σ(max) 0.000, Δ/σ (mean), 0.000. Final difference map between +0.843 and −0.524 e Å⁻³.

FIG. 7 shows a view of a molecule of Form 5 from the crystal structure showing the numbering scheme employed. Anisotropic atomic displacement ellipsoids for the non-hydrogen atoms are shown at the 50% probability level. The inter-molecular hydrogen bonds are shown as dashed lines. Hydrogen atoms are displayed with an arbitrarily small radius.

### Example 5

### Crystallization Development of Form 5

Experiments were conducted using (2S,5R)-2-(5-(3-aminopropyl)-1,3,4-oxadiazol-2-yl)-7-oxo-1,6-diazabicyclo[3.2.1]octan-6-yl hydrogen sulfate (10 mg). The solvent was added in increments and the samples heated to either 50° C. or 75° C. after each addition until a solution had been formed. The samples were then cooled to 5° C. (no specified ramp rate). Solvent was removed by pipette and the samples dried in a vacuum oven (˜5 mmHg) at 25° C. for 3 h. Yields were calculated for all samples and XRPD diffractograms collected. The samples which had been heated to 75° C. were also analysed by HPLC.

All experiments yielded Form 5 when analysed by XRPD. The results from these studies are summarized in Table 12.

TABLE 12
Results from Solvent System Experiments

| | Temperature/ | Volumes | Yield | Purity |
|---|---|---|---|---|
| Solvent | ° C. | Required | (%) | (%) |
| 95/5 water/IPA | 50 | 15 | 81 | — |
| 90/10 water/IPA | | | 81 | — |
| 80/20 water/IPA | | | 83 | — |
| 95/5 water/EG | | | 81 | — |
| 90/10 water/EG | | | 81 | — |
| 80/20 water/EG | | 20 | 79 | — |
| water | | | 76 | — |
| 95/5 water/IPA | 75 | 5 | 86 | 95.9 |
| 90/10 water/IPA | | | 83 | 96.7 |
| 80/20 water/IPA | | | 89 | 95.4 |
| water | | | 82 | 95.4 |

EG = ethylene glycol

### Scale-Up Procedure

Water/IPA (60/40, 20 vol) was used as the solvent the solvent system. The sample was heated to 60° C. at 5° C./min then held at 60° C. for 5 minutes to ensure complete dissolution was achieved. The sample was then cooled to 5° C. at 0.5° C./min. A stir speed of 500 rpm was employed throughout the experiment. The samples were filtered cold under suction and then dried in a vacuum oven at 25° C. overnight. A second crop of material was obtained from the mother liquor, which was analyzed separately from the main batch.

### Example 6

### Synergy MIC (sMIC) Assay

The synergy MIC (sMIC) assay determines the concentration of the BLI required to potentiate the activity of a fixed concentration of a β-lactam antibiotic against β-lactamase producing bacterial strains. The experimental protocol was performed according to Clinical and Laboratory Standards Institute (CLSI) guidelines with modifications as described below (CLSI guidelines can be derived from the CLSI document M07-A9 published in January 2012: “Methods for Dilution Antimicrobial Susceptibility Tests for Bacteria That Grow Aerobically; Approved Standard-Ninth Edition”). The assay wa set-up by serially diluting the BLI across 11 of the 12 wells in each row of a 96-well broth microdilution assay plate, adding the β-lactam at a fixed concentration to all wells in the assay plate, inoculating the assay plate with bacterial strains, and determining the lowest concentration of BLI required to inhibit overnight bacterial growth. Bacterial growth in the 12^{th} well of the assay plate, which contained the β-lactam at a fixed concentration but did not contain any BLI, demonstrates that the bacterial strains were resistant to the β-lactam antibiotic (e.g ceftolozane) at the fixed concentration of 4 μg/mL.

To prepare for MIC testing, frozen glycerol stocks of clinical isolates (Klebsiella pneumoniae, Escherichia coli, Enterobacter spp, Citrobacter spp, or Pseudomonas aeruginosa) were used to streak for isolated colonies on rich, non-selective, tryptic soy agar containing 5% sheep's blood (TSAB). Frozen glycerol stocks of laboratory engineered, isogenic E. coli strains, which contain cloned β-lactamase expressing plasmids were used to streak for isolated colonies on rich, selective LB agar supplemented with 25 μg/mL tetracycline to maintain the plasmid. All strains were incubated at 37° C. for 18-24 hrs.

On the day of testing, primary cultures were started by scraping off 5-10 colonies from the TSAB plates containing clinical strains or the tetracycline supplemented LB plates containing engineered strains. The clinical strain material was suspended in ˜5 mL of cation adjusted Mueller Hinton Broth (CAMHB) in 14 mL culture tubes. The engineered strain material was suspended in CAMHB (supplemented with tetracycline at 25 μg/mL) in 14 mL culture tubes. All strains were incubated at 37° C. with aeration (200 rpm) for ≧2 hrs until the OD600 was ≧0.1.

The two compound components of the assay were each prepared in CAMHB and added to the 96-well broth microdilution assay plates. 50 μL of the BLI was added to each well of the assay plate in 2-fold dilutions with final concentrations ranging from 128 to 0.13 μg/mL. 25 μL of the β-lactam was added to all wells in the broth microdilution plates at a final concentration of 4 μg/mL. Inoculum cultures were prepared by standardizing the primary cultures to OD600=0.1 and then adding 20 μL of the adjusted primary culture per 1 mL CAMHB for clinical strains or CAMHB (supplemented with tetracycline at 100 μg/mL) for isogenic strains, so that the final inoculum density was ˜10⁵ colony forming units per milliliter. Diluted inoculum cultures were used to inoculate 25 μL per well in 96-well broth microdilution assay plates. The final volume of each well was 100 μL and contained a BLI at different concentrations, a β-lactam at 4 μg/mL concentration, the bacterial culture at an OD600 of approximately 0.001 and when necessary tetracycline at 25 ug/mL.

Interpreting the sMIC Data:

Plates were incubated for 18-20 hours at 37° C. with aeration (200 rpm). Following incubation, growth was confirmed visually placing plates over a viewing apparatus (stand with a mirror underneath) and then OD600 was measured using a SpectraMax 340PC384 plate reader (Molecular Devices, Sunnyvale, Calif.). Growth was defined as turbidity that could be detected with the naked eye or achieving minimum OD600 of 0.1. sMIC values were defined as the lowest concentration producing no visible turbidity.

The sMIC values represent the amount of BLI required to potentiate the activity of 4 μg/ml of CXA-101 (Ceftolozane) or ceftazidime to inhibit the growth of the β-lactamase producing bacteria.

sMIC values of (2S,5R)-2-(5-(3-aminopropyl)-1,3,4-oxadiazol-2-yl)-7-oxo-1,6-diazabicyclo[3.2.1]octan-6-yl hydrogen sulfate are shown in Table A.

TABLE A
Synergy MIC (sMIC) Against a Panel
of Strains Expressing β-lactamases

| β-Lactamase | Bkgd | Sp | β-Lactam (4 μg/mL) | ψ |
|---|---|---|---|---|
| none | isogenic | Eco | none | D |
| KPC-2 | isogenic | Eco | CXA-101 | B |
| OXA-15 | isogenic | Eco | CXA-101 | B |
| CTX-M-15 | isogenic | Eco | CXA-101 | A |
| SHV-12 | isogenic | Eco | CXA-101 | B |
| P99 | isogenic | Eco | CXA-101 | B |
| KPC-2 | clinical | Kpn | CXA-101 | B |
| KPC-2 | clinical | Pae | CXA-101 | B |

AA = <0.25 μg/mL;
A = 0.25-0.5 μg/mL;
B = 1-2 μg/mL;
C = 4-8 μg/mL;
D = 16-32 μg/mL;
E = 64 μg/mL;
F = ≧128 μg/mL;
CXA-101 is Ceftolozane;
Eco is Escherichia coli;
Kpn is Klebsiella pneumonia;
Pae is Pseudomonas aeruginosa.
Ψ = (2S,5R)-2-(5-(3-aminopropyl)-1,3,4-oxadiazol-2-yl)-7-oxo-1,6-diazabicyclo[3.2.1]octan-6-yl hydrogen sulfate.

### Example 7

### Standard BLI Potentiation MIC Assay in Combination with Ceftolozane

The ability of compounds to potentiate the activity of β-lactams was demonstrated by determining the minimum inhibitory concentrations (MIC) of β-lactam and BLI compound combinations against various β-lactamase producing bacterial strains using the broth microdilution method. The experimental protocol was performed according to Clinical and Laboratory Standards Institute (CLSI) guidelines with modifications as described below (CLSI guidelines can be derived from the CLSI document M07-A9 published in January 2012: “Methods for Dilution Antimicrobial Susceptibility Tests for Bacteria That Grow Aerobically; Approved Standard-Ninth Edition”).

To prepare for MIC testing, frozen glycerol stocks of clinical isolates (Klebsiella pneumoniae, Escherichia coli, Enterobacter spp, Citrobacter spp, or Pseudomonas aeruginosa) were used to streak for isolated colonies on rich, non-selective, tryptic soy agar containing 5% sheep's blood (TSAB). Frozen glycerol stocks of laboratory engineered, isogenic E. coli strains, which contain cloned β-lactamase expressing plasmids were used to streak for isolated colonies on rich, selective LB agar supplemented with 25 μg/mL tetracycline to maintain the plasmid. All strains were incubated at 37° C. for 18-24 hrs.

On the day of testing, primary cultures were started by scraping off 5-10 colonies from the TSAB plates containing clinical strains or the tetracycline supplemented LB plates containing engineered strains. The clinical strain material was suspended in ˜5 mL of cation adjusted Mueller Hinton Broth (CAMHB) in 14 mL culture tubes. The engineered strain material was suspended in CAMHB (supplemented with 25 μg/mL tetracycline) in 14 mL culture tubes. All strains were incubated at 37° C. with aeration (200 rpm) for ˜2 hrs until the optical density at 600 nm (OD600) was ≧0.1.

The two compound components of the assay were each diluted in CAMHB and added to the 96-well broth microdilution assay plates. 50 μL of the β-lactam was added to each well of the assay plate in 2-fold dilutions with final concentrations ranging from 128 to 0.13 μg/mL. 25 μL of the BLI compound was added to all wells in the broth microdilution plates at a final concentration of 4 μg/mL. Inoculum cultures were prepared by standardizing the primary cultures to OD600=0.1 and then adding 20 μL of the adjusted primary culture per 1 mL CAMHB for clinical strains or CAMHB (supplemented with tetracycline at 100 μg/mL) for engineered strains, so that the final inoculum density was ˜10⁵ colony forming units per milliliter. Diluted inoculum cultures were used to inoculate 25 μL per well in 96-well broth microdilution assay plates. The final volume of each well was 100 μL and contained a β-lactam at different concentrations, a BLIcompound at 4 μg/mL concentration, the bacterial culture at an OD600 of approximately 0.001 and when necessary tetracycline at 25 μg/mL.

Plates were incubated for 18-20 hours at 37° C. with aeration (200 rpm). Following incubation, growth was confirmed visually placing plates over a viewing apparatus (stand with a mirror underneath) and then OD600 was measured using a SpectraMax 340PC384 plate reader (Molecular Devices, Sunnyvale, Calif.). Growth was defined as turbidity that could be detected with the naked eye or achieving minimum OD600 of 0.1. MIC values were defined as the lowest concentration producing no visible turbidity.

MIC values (2S,5R)-2-(5-(3-aminopropyl)-1,3,4-oxadiazol-2-yl)-7-oxo-1,6-diazabicyclo[3.2.1]octan-6-yl hydrogen sulfate are shown in Table B.

TABLE B
Standard BLI Potentiation MIC Assay in Combination
with Ceftolozane Against a Panel of Isogenic and
Clinical Strains Expressing β-Lactamases

| Strain # | β-Lactamase | Bkgd | No BLI | ψ |
|---|---|---|---|---|
| Eco.2806 | KPC-2 | isogenic | E | A |
| Pae.2808 | KPC-2 | clinical | E | B |
| Kpn.2478 | KPC-3, TEM+ | clinical | E | AA |
| Kpn.2490 | KPC-3, SHV+, TEM+ | clinical | E | AA |
| Kpn.2783 | CTX-M-15, SHV+, TEM+ | clinical | E | B |
| Kpn.571 | TEM-26 | clinical | D | AA |
| Pae.2885 | AmpC | clinical | B | A |
| Cfr.568 | AmpC | clinical | E | A |
| Ecl.569 | AmpC | clinical | E | A |
| Kpn.2914 | KPC-2, SHV+ | clinical | D | A |
| Kpn.2913 | KPC-2, SHV+ | clinical | D | AA |
| Kpn.2917 | KPC-2, SHV+ | clinical | D | AA |
| Kpn.2918 | KPC-3, SHV+, TEM+ | clinical | E | B |
| Kpn.2909 | KPC-3, SHV+, TEM+ | clinical | E | AA |
| Eco.2711 | KPC | clinical | D | AA |
| Eco.2781 | KPC-2, TEM+ | clinical | C | AA |
| Kpn.2926 | CTX-M-15, OXA-48 | clinical | E | A |
| Pae.2757 | AmpC over-expn | clinical | C | B |
| Pae.2863 | AmpC de-repress | clinical | C | B |
| Eco.2843 | DHA-1 | isogenic | E | AA |
| Eco.2491 | CMY-2 | clinical | D | AA |
| Eco.2902 | Aba-ADC-33 | isogenic | E | A |
| Eco.2840 | KPC-4 | isogenic | E | C |
| Eco.2845 | OXA-15 | isogenic | E | B |

| | | |
|---|---|---|
| MIC90 | E | B |
| MIC50 | E | A |

AA = <0.25 μg/mL;
A = 0.25-0.5 μg/mL;
B = 1-2 μg/mL;
C = 4-8 μg/mL;
D = 16-32 μg/mL;
E ≧64 μg/mL;
CXA-101 is Ceftolozane;
Eco is Escherichia coli;
Kpn is Klebsiella pneumonia;
Pae is Pseudomonas aeruginosa
Ψ = (2S,5R)-2-(5-(3-aminopropyl)-1,3,4-oxadiazol-2-yl)-7-oxo-1,6-diazabicyclo[3.2.1]octan-6-yl hydrogen sulfate.

1. A crystalline form of the compound of Formula (I):

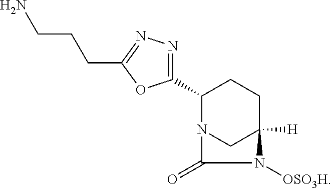

2. The crystalline form of claim 1, wherein the form is characterized by an X-ray powder diffraction pattern having four or more peaks expressed in degrees 2-theta at angles of 9.0°±0.2°, 10.0°±0.2°, 10.8°±0.2°, 13.3°±0.2°, 16.0°±0.2°, 19.7°±0.2°, 22.5°±0.2°, 28.3°±0.2°, and 29.3°±0.2°.

3. The crystalline form of claim 1, wherein the form is characterized by an X-ray powder diffraction pattern having peaks expressed in degrees 2-theta at angles of 9.0°±0.2°, 10.0°±0.2°, 16.0°±0.2°, 22.5°±0.2° and 29.3°±0.2°.

4. The crystalline form of claim 1, wherein the form is characterized by an X-ray powder diffraction pattern having peaks expressed in degrees 2-theta at angles of 9.0±0.2°, 10.0±0.2°, 14.0±0.2°, 17.9±0.2°, 20.1±0.2°, 21.0±0.2°, 22.5±0.2°, 24.3±0.2°, and 29.3±0.2°.

5. The crystalline form of claim 1, wherein the form is characterized by an X-ray powder diffraction pattern having peaks expressed in degrees 2-theta at angles of 9.0±0.2°, 10.0±0.2°, 20.1±0.2°, 21.0±0.2°, and 22.5±0.2°.

6. A pharmaceutical composition comprising the crystalline form of claim 1 and optionally a pharmaceutically acceptable carrier or diluent.

7. The pharmaceutical composition of claim 6, further comprising at least one β-lactam antibiotic.

8. (canceled)

9. The pharmaceutical composition of claim 7, wherein the cephalosporin is Ceftolozane.

10. (canceled)

11. (canceled)

12. (canceled)

13. A method of treating or preventing a bacterial infection comprising administering to a subject in need thereof a therapeutically-effective amount of a β-lactam antibiotic in conjunction with the crystalline form of claim 1.

14. The method of claim 13, wherein the bacterial infection is caused by bacteria that produce a class A, class C or class D β-lactamase.

15. (canceled)

16. The method of claim 13, wherein the bacterial infection is caused by bacteria selected from Acinetobacter spp., Acinetobacter baumannii, Citrobacter spp., Escherichia spp., Escherichia coli, Haemophilus influenzae, Morganella morganii, Pseudomonas aeruginosa, Klebsiella spp., Klebsiella pneumoniae, Enterobacter spp., Enterobacter cloacae, Enterobacter aerogenes Pasteurella spp., Proteus spp., Proteus mirabilis, Serratia spp., Serratia marcescens, and Providencia spp.

17. The method of claim 13, wherein the bacterial infection is caused by bacteria that produce a KPC-2 or KPC-3 β-lactamase.

18. The method of claim 13, wherein the bacterial infection is caused by bacteria that produce an OXA-15 β-lactamase.

19. The method of claim 13, wherein the bacterial infection is caused by β-lactam resistant bacteria.

20. A method of treating a bacterial infection in a subject in need thereof, comprising the steps of:
(I) administering to the subject a therapeutically-effective amount of a β-lactam antibiotic; then
(II) administering to the subject a crystalline form of Formula (I); or
(I) administering to the subject a crystalline form of Formula (I); then
(II) administering to the subject a therapeutically-effective amount of a β-lactam antibiotic.

21. (canceled)

22. A method of inhibiting β-lactamase comprising administering to a subject the crystalline form of claim 1.

23. The method of claim 13, wherein the crystalline form is characterized by an X-ray powder diffraction pattern having four or more peaks expressed in degrees 2-theta at angles of 9.0°±0.2°, 10.0°±0.2°, 10.8°±0.2°, 13.3°±0.2°, 16.0°±0.2°, 19.7°±0.2°, 22.5°±0.2°, 28.3°±0.2°, and 29.3°±0.2°.

24. The method of claim 13, wherein the crystalline form is characterized by an X-ray powder diffraction pattern having four or more peaks expressed in degrees 2-theta at angles of 9.0±0.2°, 10.0±0.2°, 14.0±0.2°, 17.9±0.2°, 20.1±0.2°, 21.0±0.2°, 22.5±0.2°, 24.3±0.2°, and 29.3±0.2°.

25. (canceled)

26. A method of making a crystalline form of the compound of Formula (I), comprising the steps of:
(I) combining (2S,5R)-2-(5-(3-aminopropyl)-1,3,4-oxadiazol-2-yl)-7-oxo-1,6-diazabicyclo[3.2.1]octan-6-yl hydrogen sulfate and a solvent, such that a solution of (2S,5R)-2-(5-(3-aminopropyl)-1,3,4-oxadiazol-2-yl)-7-oxo-1,6-diazabicyclo[3.2.1]octan-6-yl hydrogen sulfate is formed; and
(II) evaporating the solvent, such that a crystalline solid form of the compound of Formula (I) is formed; or
(I) combining (2S,5R)-2-(5-(3-aminopropyl)-1,3,4-oxadiazol-2-yl)-7-oxo-1,6-diazabicyclo[3.2.1]octan-6-yl hydrogen sulfate and a solvent, such that a first solution of (2S,5R)-2-(5-(3-aminopropyl)-1,3,4-oxadiazol-2-yl)-7-oxo-1,6-diazabicyclo[3.2.1]octan-6-yl hydrogen sulfate is formed;
(II) combining the first solution with a second solvent; and
(III) crystallizing the compound of Formula (I).

27. (canceled)

28. The method of claim 26, wherein the crystalline form of the compound of Formula (I) is characterized by an X-ray powder diffraction pattern having four or more peaks expressed in degrees 2-theta at angles of 9.0°±0.2°, 10.0°±0.2°, 10.8°±0.2°, 13.3°±0.2°, 16.0°±0.2°, 19.7°±0.2°, 22.5°±0.2°, 28.3°±0.2°, and 29.3°±0.2°.

29. The method of claim 26, wherein the crystalline form of the compound of Formula (I) is characterized by an X-ray powder diffraction pattern having four or more peaks expressed in degrees 2-theta at angles of 9.0±0.2°, 10.0±0.2°, 14.0±0.2°, 17.9±0.2°, 20.1±0.2°, 21.0±0.2°, 22.5±0.2°, 24.3±0.2°, and 29.3±0.2°.

30. A crystalline form of the compound of Formula (I): 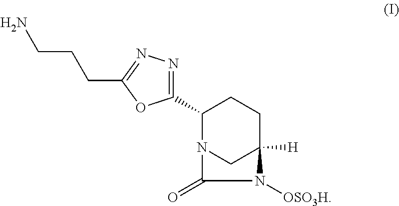
characterized by an X-ray powder diffraction pattern having peaks expressed in degrees 2-theta at angles of 9.0°±0.2°, 10.0°±0.2°, 16.0°±0.2°, 22.5°±0.2° and 29.3°±0.2°.

31. The crystalline form of claim 30, characterized by an X-ray powder diffraction pattern having peaks expressed in degrees 2-theta at angles of 9.0°±0.2°, 10.0°±0.2°, 10.8°±0.2°, 13.3°±0.2°, 16.0°±0.2°, 19.7°±0.2°, 22.5°±0.2°, 28.3°±0.2°, and 29.3°±0.2°.

32. The crystalline form of claim 30, characterized by a differential scanning calorimetry thermogram having an endotherm peak at about 129° C.

33. The crystalline form of claim 30, characterized by a differential scanning calorimetry thermogram having an exotherm peak at about 195° C.

34. The crystalline form of claim 30, characterized by a thermogravimetry curve showing a first endotherm between 25° C. and 125° C.

35. The crystalline form of claim 34, wherein the thermogravimetry curve shows a weight loss of about 5% corresponding to the first endotherm.

36. The crystalline form of claim 30, characterized by a thermogravimetry curve showing a second endotherm between 125° C. and 145° C.

37. The crystalline form of claim 36, wherein the thermogravimetry curve shows a weight loss of about 5.5% corresponding to the second endotherm.

38. The crystalline form of claim 30, obtained by a method comprising the steps of:
(I) combining (2S,5R)-2-(5-(3-aminopropyl)-1,3,4-oxadiazol-2-yl)-7-oxo-1,6-diazabicyclo[3.2.1]octan-6-yl hydrogen sulfate and a solvent, such that a solution of (2S,5R)-2-(5-(3-aminopropyl)-1,3,4-oxadiazol-2-yl)-7-oxo-1,6-diazabicyclo[3.2.1]octan-6-yl hydrogen sulfate is formed; and
(II) evaporating the solvent, such that a crystalline solid form of the compound of Formula (I) is formed; or
(I) combining (2S,5R)-2-(5-(3-aminopropyl)-1,3,4-oxadiazol-2-yl)-7-oxo-1,6-diazabicyclo[3.2.1]octan-6-yl hydrogen sulfate and a solvent, such that a first solution of (2S,5R)-2-(5-(3-aminopropyl)-1,3,4-oxadiazol-2-yl)-7-oxo-1,6-diazabicyclo[3.2.1]octan-6-yl hydrogen sulfate is formed;
(II) combining the first solution with a second solvent; and
(III) crystallizing the compound of Formula (I).
